SEQUENCE LISTING (8 sequences)

SEQ ID NO: 1 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 12 aa, synthetic construct
RASQRVSSSYLA

SEQ ID NO: 3 - protein, 7 aa, synthetic construct
DASSRAT

SEQ ID NO: 4 - protein, 9 aa, synthetic construct
QQYGSLPWT

SEQ ID NO: 5 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 6 - protein, 10 aa, synthetic construct
GFTFSRYWMS

SEQ ID NO: 7 - protein, 17 aa, synthetic construct
NIKQDGSEKYYVDSVKG

SEQ ID NO: 8 - protein, 12 aa, synthetic construct
EGGWFGELAFDY

(19) United States
(12) Patent Application Publication
(10) Pub. No.: US 2026/0284189 A1
(43) Pub. Date: Sep. 24, 2026

(54) TREATMENT OF NON-SMALL CELL LUNG CANCER

(71) Applicant: ASTRAZENECA AB, SODERTALJE (SE)

(72) Inventors: Norah SHIRE, Wilmington, DE (US); Phillip DENNIS, Wilmington, DE (US)

(21) Appl. No.: 19/473,913

(22) PCT Filed: Oct. 17, 2024
(86) PCT No.: PCT/EP2024/059945

§ 371 Date: Oct. 9, 2025

Related U.S. Application Data
(60) Provisional application No. 63/496,200, filed on Apr. 14, 2023.
(60) Provisional application No. 63/509,408, filed on Jun. 21, 2023.

(51) Int. Cl. A61K 39/395; A61K 31/282; A61K 31/337; A61K 31/519; A61K 31/7068; A61K 33/243; A61K 39/00; A61P 35/00; C07K 16/28

(52) U.S. Cl. CPC A61K 39/3955; A61K 31/282; A61K 31/337; A61K 31/519; A61K 31/7068; A61K 33/243; A61P 35/00; C07K 16/2827; A61K 2039/505; C07K 2317/21

(57) ABSTRACT

The disclosure generally relates to methods and compositions for treating patients with resectable non-small cell lung cancer.

### CROSS-REFERENCE TO RELATED APPLICATIONS

This application claims the benefit of priority to U.S. Provisional Application Nos: 63/496,200, filed Apr. 14, 2023, and to 63/509,408, filed Jun. 21, 2023, each of which is incorporated by reference herein in its entirety for all purposes.

### FIELD OF THE DISCLOSURE

The present disclosure generally relates to methods and compositions for treating patients with non-small cell lung cancer using durvalumab and platinum-based chemotherapy.

### SEQUENCE LISTING

This application contains a sequence listing which is submitted electronically and is hereby incorporated by reference in its entirety. The sequence listing submitted herewith is contained in the XML filed created May 26, 2023 entitled “23-0535-US-PRO2_SequenceListing.xml” and is 8,192 bytes in size.

### BACKGROUND OF THE DISCLOSURE

Lung cancer is the leading cause of cancer-related death worldwide, with non-small-cell lung cancer (NSCLC) accounting for over 80% of cases. Approximately 25-30% of patients present with resectable disease at diagnosis, a proportion that is expected to increase with growing use of lung cancer screening programs. Surgery remains the primary curative-intent treatment for eligible patients with early-stage NSCLC. Despite this, many patients experience tumor recurrence within 5 years of surgery (~30-55% depending on disease stage at diagnosis), leading to deterioration in health-related quality of life and increasing the likelihood of disease-related death. Chemotherapy, in the neoadjuvant or adjuvant setting, is a well-established treatment strategy, but offers only a modest improvement in 5-year survival of approximately 5% versus surgery alone.

Lung cancer has been the most common cancer in the world for several decades, with an estimated 1.8 million new cases in 2012 (12.9% of all new cancers), and was also the most common cause of death from cancer in 2012, with 1.6 million deaths (19.4% of cancer deaths; GLOBOCAN 2012). Non-small cell lung cancer (NSCLC) represents 80% to 85% of all lung cancers (Pisters and Le Chevalier, “Adjuvant chemotherapy in completely resected non-small-cell lung cancer: J Clin Oncol. 2005 May 10; 23(14):3270-8). Despite advances in the diagnosis, imaging, staging, and treatment of NSCLC, the estimated 5-year overall survival (OS) for patients in Europe and the United States continues to be low (11% and 17%, respectively; D'Addario et al. “Metastatic non-small-cell lung cancer: ESMO Clinical Practice Guidelines for diagnosis, treatment and follow-up” Ann Oncol. 2010 May; 21 Suppl 5:v116-9; Howlader et al. “SEER Cancer Statistics Review, 1975-2014”, National Cancer Institute. Bethesda, MD. April 2017).

For early-stage NSCLC, the primary treatment is curative surgery. Only ~30% of patients present with Stages I to IIIA lung cancer; however, this percentage is expected to increase as a result of the implementation of lung cancer screening. Unfortunately, the 5-year survival for patients treated with surgery alone remains low, ranging from 67% (Stage IA) to 23% (Stage IIIA) (Mountain “Revisions in the international system for staging lung cancer.” Chest 1997; 111:1710-7.).

Studies of adjuvant chemotherapy in the NSCLC setting have demonstrated a modest but clinically meaningful improvement in overall survival; perioperative platinum-based chemotherapy has shown a survival rate of 5.4 percentage points higher than surgery alone, with toxicities of Grade 3 or higher observed in more than 60% of patients (Pignon et al. “Lung adjuvant cisplatin evaluation: a pooled analysis by the LACE Collaborative Group.” J C/in Oncol 2008; 26:3552-9; Wakelee et al. “Adjuvant chemotherapy with or without bevacizumab in patients with resected non-small-cell lung cancer (E1505): an open-label, multicentre, randomised, phase 3 trial.” Lancet Oncol 2017; 18:1610-23).

A number of studies have demonstrated the clinical benefit of neoadjuvant chemotherapy in early-stage NSCLC. However, despite advances, new therapies are needed to further improve the long-term prognosis for patients with NSCLC who undergo surgical resection.

### SUMMARY OF THE DISCLOSURE

The present disclosure generally relates to methods for treating resectable non-small cell lung cancer patients (NSCLC). This disclosure demonstrates that perioperative durvalumab plus neoadjuvant chemotherapy results in significant improvement of both pathological complete response (PCR) and event free survival (EFS) among patients with resectable NSCLC.

In an aspect, this disclosure provides a method of treating a patient identified as having resectable non-small cell lung cancer (R-NSCLC), the method comprising administering to the patient a combination therapy comprising durvalumab and a platinum-based chemotherapy.

In certain embodiments, the medicament is administered about every 14 to 28 days or about every 21 days. In certain embodiments, the combination therapy is administered about every 14 to 28 days for about 3 to 6 cycles. In certain embodiments, the combination therapy is administered about every 21 days (Q3W) for about 4 cycles. In certain embodiments, the method further comprises resecting the R-NSCLC after the about 3 to 6 cycles of the combination therapy. In certain embodiments, the method further comprises resecting the R-NSCLC after the about 4 cycles of the combination therapy. In certain embodiments, the method further comprises administering to the patient durvalumab about every 14 to 28 days for up to about twelve cycles after resecting the R-NSCLC. In certain embodiments, the method further comprises administering to the patient durvalumab about every 28 days (Q4W) for up to about twelve cycles after resecting the R-NSCLC.

In certain embodiments, the combination therapy comprises about 1000 to 2000 mg of durvalumab. In certain embodiments, the combination therapy comprises about 1500 mg of durvalumab.

In another aspect, this disclosure provides a method of treating a patient identified as having resectable non-small cell lung carcinoma (R-NSCLC), the method comprising
(i) administering to the patient about 1500 mg of durvalumab and a platinum-based chemotherapy about every 3 weeks (Q3W) for about 4 cycles;
(ii) removing the R-NSCLC by surgery; and then
(iii) administering to the patient about 1500 mg of durvalumab about every four weeks (Q4W) for at least about 12 weeks.

In certain embodiments of the method disclosed herein, the platinum-based chemotherapy is one or more of carboplatin, cisplatin, nedaplatin, and oxaliplatin. In certain embodiments, the platinum-based chemotherapy comprises a carboplatin area under the serum drug concentration-time curve (AUC) dose of about 5 to about 6 mg/mL/min, or a cisplatin dose of about 75 mg/m². In certain embodiments, the platinum-based chemotherapy further comprises one or more of afatinib, cetuximab, bevacizumab, erlotinib, gemcitabine, paclitaxel, pemetrexed, and vemurafenib. In certain embodiments, the platinum-based chemotherapy comprises a paclitaxel dose of about 200 mg/m², a pemetrexed dose of about 500 mg/m², or a gemcitabine dose of about 1250 mg/m².

In certain embodiments of the methods disclosed herein, the R-NSCLC is a squamous cell carcinoma. In certain embodiments, the platinum-based chemotherapy comprises: (i) carboplatin and paclitaxel; (ii) cisplatin and gemcitabine; and/or (iii) carboplatin and gemcitabine.

In certain embodiments of the method disclosed herein, the R-NSCLC is a non-squamous cell carcinoma. In certain embodiments, the platinum-based chemotherapy comprises: (i) carboplatin and pemetrexed; and/or (ii) cisplatin and pemetrexed.

In certain embodiments of the methods disclosed herein, the patient does not have an EGFR mutation and/or an ALK translocation.

In certain embodiments of the methods disclosed herein, resecting the R-NSCLC occurs within about 10 weeks after the about 3 to 6 cycles of the combination therapy.

In certain embodiments of the methods disclosed herein, the methods further comprise a post-operative radiation therapy. In certain embodiments, the post-operative radiation therapy starts within about 8 weeks after resecting the R-NSCLC. In certain embodiments, durvalumab starts within about 3 weeks from the end of the post-operative radiation therapy.

In certain embodiments of the methods disclosed herein, the method results in one or more of. (i) improvement in event-free survival (EFS) of at least about 13 months; (ii) improvement in pathological complete response (pCR) by at least about 13%; and (iii) improvement in major pathological response (mPR) by at least about 21%. In certain embodiments, the method results in improvement in one or more of EFS, pCR, mPR, DFS, and OS compared to a standard of care. In certain embodiments, the method results in improvement in one or more of EFS, pCR, mPR, DFS, and OS compared to a platinum-based chemotherapy, or platinum-based chemotherapy following or preceding surgery.

In an aspect, this disclosure provides a combination therapy comprising durvalumab and a platinum-based chemotherapy for use in treating resectable non-small cell lung cancer (R-NSCLC) in a patient in need thereof.

In certain embodiments, the combination therapy is administered about every 14 to 28 days or about every 21 days. In certain embodiments, the combination therapy is administered about every 14 to 28 days for about 3 to 6 cycles. In certain embodiments, the combination therapy is administered about every 21 days (Q3W) for about 4 cycles. In certain embodiments, the combination therapy further comprises resecting the R-NSCLC after the about 3 to 6 cycles of the combination therapy. In certain embodiments, the combination therapy further comprises resecting the R-NSCLC after the about 4 cycles of the combination therapy. In certain embodiments, the combination therapy further comprises administering to the patient durvalumab about every 14 to 28 days for up to about twelve cycles after resecting the R-NSCLC. In certain embodiments, the combination therapy further comprises administering to the patient durvalumab about every 28 days (Q4W) for up to about twelve cycles after resecting the R-NSCLC.

In certain embodiments, the combination therapy comprises about 1000 to 2000 mg of durvalumab. In certain embodiments, the combination therapy comprises about 1500 mg of durvalumab.

In another aspect, this disclosure provides a combination therapy for use in treating a patient identified as having resectable non-small cell lung carcinoma (R-NSCLC), wherein the combination therapy comprises: administering to the patient about 1500 mg of durvalumab and a platinum-based chemotherapy about every 3 weeks (Q3W) for about 4 cycles; and then administering to the patient about 1500 mg of durvalumab about every four weeks (Q4W) for at least about 12 weeks after removal of the R-NSCLC by surgery.

In certain embodiments of the combination therapy, the platinum-based chemotherapy is one or more of carboplatin, cisplatin, nedaplatin, and oxaliplatin. In certain embodiments, the platinum-based chemotherapy comprises a carboplatin area under the serum drug concentration-time curve (AUC) dose of about 5 to about 6 mg/mL/min, or a cisplatin dose of about 75 mg/m². In certain embodiments, the platinum-based chemotherapy further comprises one or more of afatinib, cetuximab, bevacizumab, erlotinib, gemcitabine, paclitaxel, pemetrexed, and vemurafenib. In certain embodiments of the combination therapy, the platinum-based chemotherapy comprises a paclitaxel dose of about 200 mg/m², a pemetrexed dose of about 500 mg/m², or a gemcitabine dose of about 1250 mg/m2.

In certain embodiments of the combination therapy, the R-NSCLC is a squamous cell carcinoma. In certain embodiments, the platinum-based chemotherapy comprises: (i) carboplatin and paclitaxel; (ii) cisplatin and gemcitabine; and/or (iii) carboplatin and gemcitabine.

In certain embodiments of the combination therapy, the R-NSCLC is a non-squamous cell carcinoma. In certain embodiments, the platinum-based chemotherapy comprises: (i) carboplatin and pemetrexed; and/or (ii) cisplatin and pemetrexed.

In certain embodiments of the combination therapy, the patient does not have an EGFR mutation and/or an ALK translocation.

In certain embodiments of the combination therapy, resecting the R-NSCLC occurs within about 10 weeks after the about 3 to 6 cycles of the combination therapy.

In certain embodiments, the combination therapy further comprises a post-operative radiation therapy. In certain embodiments, the post-operative radiation therapy starts within about 8 weeks after resecting the R-NSCLC. In certain embodiments, durvalumab starts within about 3 weeks from the end of the post-operative radiation therapy.

In certain embodiments of the combination therapy, the use of the combination therapy results in one or more of: (i) improvement in event-free survival (EFS) of at least about 13 months; (ii) improvement in pathological complete response (pCR) by at least about 13%; and (iii) improvement in major pathological response (mPR) by at least about 21%. In certain embodiments, the use of the combination therapy results in improvement in one or more of EFS, pCR, mPR, DFS and OS compared to a standard of care. In certain embodiments, the use of the combination therapy results in improvement in one or more of EFS, pCR, mPR, DFS and OS compared to a platinum-based chemotherapy or platinum-based chemotherapy following or preceding surgery.

In an aspect, this disclosure provides the use of a combination therapy comprising durvalumab and a platinum-based chemotherapy for the manufacture of a medicament for treating resectable non-small cell lung cancer (R-NSCLC) in a patient in need thereof.

In certain embodiments, the medicament is administered about every 14 to 28 days or about every 21 days. In certain embodiments, the medicament is administered about every 14 to 28 days for about 3 to 6 cycles. In certain embodiments, the medicament is administered about every 21 days (Q3W) for about 4 cycles. In certain embodiments, the use of the medicament further comprising resecting the R-NSCLC after the about 3 to 6 cycles of the combination therapy. In certain embodiments, the use of the medicament further comprises resecting the R-NSCLC after the about 4 cycles of the combination therapy. In certain embodiments, the use of medicament further comprises administering to the patient durvalumab about every 14 to 28 days for up to about twelve cycles after resecting the R-NSCLC. In certain embodiments, the use of medicament further comprises administering to the patient durvalumab about every 28 days (Q4W) for up to twelve cycles after resecting the R-NSCLC.

In certain embodiments of the use of the medicament, the medicament comprises about 1000 to 2000 mg of durvalumab. In certain embodiments, the medicament comprises about 1500 mg of durvalumab.

In another aspect, this disclosure provides the use of a durvalumab and a platinum-based chemotherapy in the manufacture of a medicament for treating resectable non-small cell lung cancer (R-NSCLC) in a combination therapy, wherein the combination therapy comprises: administering to the patient about 1500 mg of durvalumab and a platinum-based chemotherapy about every 3 weeks (Q3W) for about 4 cycles; removing the R-NSCLC by surgery; and then administering to the patient about 1500 mg of durvalumab about every four weeks (Q4W) for at least about 12 weeks.

In certain embodiments of the use of the medicament, the platinum-based chemotherapy is one or more of carboplatin, cisplatin, nedaplatin, and oxaliplatin. In certain embodiments, the platinum-based chemotherapy comprises a carboplatin area under the serum drug concentration-time curve (AUC) dose of about 5 to about 6 mg/mL/min, or a cisplatin dose of about 75 mg/m². In certain embodiments of the use of the medicament, the platinum-based chemotherapy further comprises one or more of afatinib, cetuximab, bevacizumab, erlotinib, gemcitabine, paclitaxel, pemetrexed, and vemurafenib. In certain embodiments of the use of the medicament, the platinum-based chemotherapy comprises a paclitaxel dose of about 200 mg/m², a pemetrexed dose of about 500 mg/m², or a gemcitabine dose of about 1250 mg/m².

In certain embodiments of the use of the medicament, the R-NSCLC is a squamous cell carcinoma. In certain embodiments, the platinum-based chemotherapy comprises: (i) carboplatin and paclitaxel; (ii) cisplatin and gemcitabine; and/or (iii) carboplatin and gemcitabine.

In certain embodiments of the use of the medicament, the R-NSCLC is a non-squamous cell carcinoma. In certain embodiments, the platinum-based chemotherapy comprises: (i) carboplatin and pemetrexed; and/or (ii) cisplatin and pemetrexed.

In certain embodiments of the use of the medicament, the patient does not have an EGFR mutation and/or an ALK translocation.

In certain embodiments of the use of the medicament, resecting the R-NSCLC occurs within about 10 weeks after the about 3 to 6 cycles of the combination therapy.

In certain embodiments of the use of the medicament, the use further comprising a post-operative radiation therapy. In certain embodiments, the post-operative radiation therapy starts within about 8 weeks after resecting the R-NSCLC. In certain embodiments, durvalumab starts within about 3 weeks from the end of the post-operative radiation therapy.

In certain embodiments of the use of the medicament, the use of the combination therapy results in one or more of: (i) improvement in event-free survival (EFS) of at least about 13 months; (ii) improvement in pathological complete response (pCR) by at least about 13%; and (iii) improvement in major pathological response (mPR) by at least about 21%. In certain embodiments, the use of the combination therapy results in improvement in one or more of EFS, pCR, mPR, DFS, and OS compared to a standard of care. In certain embodiments, the use of the combination therapy results in improvement in one or more of EFS, pCR, and mPR, DFS, and OS compared to a platinum-based chemotherapy, or platinum-based chemotherapy following or preceding surgery.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS/FIGURES

The accompanying drawings are included to provide a further understanding of the methods and compositions of the disclosure. The drawings illustrate one or more embodiments of the disclosure, and together with the description serve to explain the principles and operation of the disclosure.

FIG. 1A shows the study design for a phase 3, global, randomized, double-blind, placebo-controlled study according to the disclosure.*The protocol was amended while enrollment was ongoing to exclude (1) patients with tumors classified as T4 for any reason other than size; (2) patients with planned pneumonectomies; and (3) patients with documented EGFR/ALK aberrations. †Ventana SP263 immunohistochemistry assay. ‡Choice of CT regimen determined by histology and at the investigator's discretion. For non-squamous: cisplatin+pemetrexed, or carboplatin+pemetrexed. For squamous: carboplatin+paclitaxel or cisplatin+gemcitabine (or carboplatin+gemcitabine for patients who have comorbidities or who are unable to tolerate cisplatin per the investigator's judgment). § Post-operative radiotherapy (PORT) was permitted where indicated per local guidance. ¶ All efficacy analyses reported herein were performed on the mITT population, which includes all randomized patients who did not have documented EGFR/ALK aberrations. AJCC, American Joint Committee on Cancer; BICR, blinded independent central review; DFS, disease-free survival; EFS, event-free survival; mITT, modified intent-to-treat; MPR, major pathologic response; pCR, pathologic complete response.

FIG. 1B shows the CONSORT Flow Diagram. *Signed informed consent received. †Efficacy endpoints were analyzed based on a modified population that excludes patients with documented EGFR/ALK aberrations. ‡The safety analysis set includes all randomized patients who received at least one dose of any study Tx. AE, adverse event; DCO, data cut-off; ITT, intent-to-treat; PD, progressive disease; Tx, treatment.

FIG. 2 shows event-free survival (EFS) according to Blinded Independent Central Review (BICR) in the Modified Intent-to-treat (mITT) population. Data cut-off of Nov. 10, 2022 (N=740). The graph shows EFS among the patients in the modified intent-to-treat population (i.e., all randomized patients without documented EGFR/ALK aberrations). EFS was defined as the time from randomization to the earliest of: (i) progressive disease that precludes surgery; (ii) progressive disease discovered and reported by the investigator upon attempting surgery that prevents completion of surgery; (iii) local or distant recurrence using blinded independent central review according to Response Evaluation Criteria in Solid Tumors version 1.1; or (iv) death from any cause. Median EFS follow-up in censored patients was 11.7 months (range: 0.0-46.1); EFS maturity: 31.9%. The P-value was calculated using a stratified log rank test with the significance boundary=0.009899 (based on total 5% alpha), calculated using a Lan-DeMets alpha spending function with O'Brien Fleming boundary. CI, confidence interval; D, durvalumab; eCRF, electronic case report form; EFS, event-free survival; HR, hazard ratio; NR, not reached; PBO, placebo.

FIG. 3 shows event-free survival (EFS) according to Blinded Independent Central Review (BICR) in the Modified Intent-to-treat (mITT) population. Data cut-off of Nov. 10, 2022 (N=740). The forest plot of EFS in prespecified patient subgroups (in which the size of the circles is proportional to the number of events for each subgroup and the horizontal bars represent the 95% CIs). EFS was defined as the time from randomization to the earliest of: (i) progressive disease that precludes surgery; (ii) progressive disease discovered and reported by the investigator upon attempting surgery that prevents completion of surgery; (iii) local or distant recurrence using blinded independent central review according to Response Evaluation Criteria in Solid Tumors version 1.1; or (iv) death from any cause. *Race was self-reported per the eCRF. †Determined using the Ventana SP263 immunohistochemistry assay. AJCC, American Joint Committee on Cancer; CI, confidence interval; D, durvalumab; ECOG PS, Eastern Cooperative Oncology Group performance status; eCRF, electronic case report form; EFS, event-free survival; HR, hazard ratio; NR, not reached; PBO, placebo; PD-L1, programmed cell death-ligand 1; TC tumor cell.

FIG. 4A shows Event-free Survival according to Blinded Independent Central Review by planned neoadjuvant platinum agent in the modified Intent-to-treat population (prespecified subgroup analysis). There was flexibility in the choice of neoadjuvant chemotherapy combinations*−clear and consistent EFS benefit was observed regardless of the planned platinum agent. Median and landmark estimates calculated using the Kaplan-Meier method. HRs calculated using an unstratified Cox proportional hazards model. *Choice of CT regimen determined by histology and at the investigator's discretion. For non-squamous: cisplatin+pemetrexed, or carboplatin+pemetrexed. For squamous: carboplatin+paclitaxel, or cisplatin+gemcitabine (or carboplatin+gemcitabine for patients who have comorbidities or who are unable to tolerate cisplatin per the investigator's judgment). †HR<1 favors the D arm versus the PBO arm. Results show a clear and consistent EFS benefit was observed regardless of the planned platinum agent. CI, confidence interval; D, durvalumab; EFS, event-free survival; HR, hazard ratio; PBO, placebo.

FIG. 4B shows event-free survival according to Blinded Independent Central Review by disease stage in the modified Intent-to-treat population (prespecified subgroup analysis). Data cut-off of Nov. 10, 2022 (N=740). Top panel shows EFS among the subgroup of patients with stage II disease, and lower panel shows EFS among the subgroup of patients with stage III disease (at study baseline). EFS was defined as the time from randomization to the earliest of: (i) progressive disease that precludes surgery; (ii) progressive disease discovered and reported by the investigator upon attempting surgery that prevents completion of surgery; (iii) local or distant recurrence using blinded independent central review according to Response Evaluation Criteria in Solid Tumors version 1.1; or (iv) death from any cause. CI, confidence interval; D, durvalumab; EFS, event-free survival; HR, hazard ratio; PBO, placebo.

FIG. 4C-4E show event-free survival according to Blinded Independent Central Review by PD-L1 tumor cell expression in the modified Intent-to-treat Population (prespecified subgroup analysis). Data cut-off of Nov. 10, 2022 (N=740). FIG. 4C shows EFS among the subgroup of patients with PD-L1 tumor cell expression<1%, FIG. 4D shows EFS among the subgroup of patients with PD-L1 tumor cell expression 1-49%, and FIG. 4E shows EFS among patients with PD-L1 tumor cell expression≥50% (at study baseline). EFS was defined as the time from randomization to the earliest of: (i) progressive disease that precludes surgery; (ii) progressive disease discovered and reported by the investigator upon attempting surgery that prevents completion of surgery; (iii) local or distant recurrence using blinded independent central review according to Response Evaluation Criteria in Solid Tumors version 1.1; or (iv) death from any cause. CI, confidence interval; D, durvalumab; EFS, event-free survival; HR, hazard ratio; PBO, placebo; PD-L1, programmed cell death-ligand-1; TC, tumor cell.

FIG. 4F-4G show event-free survival according to Blinded Independent Central Review by tumor histology in the modified Intent-to-treat population (prespecified subgroup analysis). Data cut-off of Nov. 10, 2022 (N=740). FIG. 4F shows EFS among the subgroup of patients with squamous tumor histology, and FIG. 4G shows EFS among the subgroup of patients with non-squamous tumor histology (at study baseline). EFS was defined as the time from randomization to the earliest of: (i) progressive disease that precludes surgery; (ii) progressive disease discovered and reported by the investigator upon attempting surgery that prevents completion of surgery; (iii) local or distant recurrence using blinded independent central review according to Response Evaluation Criteria in Solid Tumors version 1.1; or (iv) death from any cause. CI, confidence interval; D, durvalumab; EFS, event-free survival; HR, hazard ratio; PBO, placebo.

FIG. 5 shows final analyses of pathological response according to Central Review in the modified Intent-to-treat Population. Data cut-off of Nov. 10, 2022 (N=740). Left panel shows pCR. Right panel shows MPR. pCR was defined as a lack of viable tumor cells after complete evaluation of the resected lung cancer specimen and all sampled regional lymph nodes. MPR was defined as <10% of viable tumor cells in the lung primary tumor after complete evaluation of the resected lung cancer specimen. To be eligible for pathological assessment, patients needed to have received three cycles of neoadjuvant study treatment per the protocol followed by on-study surgery, and patients who were not evaluable were classified as non-responders. No formal statistical testing was performed at the final analysis of pathological complete response (data cut-off, Nov. 10, 2022; n=740 [data shown]); statistical significance was achieved at the interim analysis (data cut-off, Jan. 14, 2022; n=402), for which the P-values were calculated using a stratified Cochran-Mantel-Haenszel test with a significance boundary=0.000082 calculated using a Lan-DeMets alpha spending function with O'Brien Fleming boundary.

FIG. 6 shows final analyses of pathological response according to Central Review in the modified Intent-to-treat Population. Data cut-off of Nov. 10, 2022 (N=740). The forest plot is of pCR in prespecified patient subgroups (in which the size of the circles is proportional to number of patients for each subgroup, and the horizontal bars represent the 95% CIs). Statistical significance was achieved at the interim analysis (data cut-off, Jan. 14, 2022; n=402), for which the P-values were calculated using a stratified Cochran-Mantel-Haenszel test with a significance boundary=0.000082 calculated using a Lan-DeMets alpha spending function with O'Brien Fleming boundary. *Race was self-reported per the eCRF. †Determined using the Ventana SP263 immunohistochemistry assay. AJCC denotes American Joint Committee on Cancer, ECOG PS Eastern Cooperative Oncology Group performance status, eCRF electronic case report form, PD-L1 programmed cell death-ligand 1, and TC tumor cell.

FIG. 7A-7B shows interim analyses of pathological response according to central review in the modified Intent-to-treat (mITT) population. Data cut-off of Jan. 14, 2022 (N=402). FIG. 7A shows pCR and FIG. 7B shows MPR in the pCR interim analysis cohort (the first ~400 patients in the mITT population who had the opportunity to undergo surgery and complete central pathology assessment for pCR [inclusive of patients not eligible for surgery]). pCR was defined as a lack of any viable tumor cells after complete evaluation of the resected lung cancer specimen and all sampled regional lymph nodes. MPR was defined as ≤10% of viable tumor cells in the lung primary tumor after complete evaluation of the resected lung cancer specimen. To be eligible for pathological assessment, patients should have had three cycles of neoadjuvant study treatment per the protocol, and patients who were not evaluable were classified as non-responders. Statistical significance was achieved at this interim analysis (N=402). The P-values were calculated using a stratified Cochran-Mantel-Haenszel test with a significance boundary=0.000082 calculated using a Lan-DeMets alpha spending function with O'Brien Fleming boundary. CI, confidence interval; D, durvalumab; MPR, major pathological response; PBO, placebo; pCR, pathological complete response.

FIG. 8 shows subgroup analysis of major pathological response according to central review in the modified Intent-to-treat population. Data cut-off of Nov. 10, 2022 (N=740). The figure shows a forest plot of MPR in prespecified patient subgroups (in which the size of the circles is proportional to number of patients for each subgroup, and the horizontal bars represent the 95% confidence intervals). MPR was defined as ≤10% of viable tumor cells in the lung primary tumor after complete evaluation of the resected lung cancer specimen. To be eligible for pathological assessment, patients needed to have received three cycles of neoadjuvant study treatment per the protocol, and patients who were not evaluable were classified as non-responders. *Race was self-reported per the eCRF. †Determined using the Ventana SP263 immunohistochemistry assay. AJCC, American Joint Committee on Cancer; D, durvalumab; ECOG PS, Eastern Cooperative Oncology Group performance status; eCRF, electronic case report form; MPR, major pathological response; PBO, placebo; PD-L1, programmed cell death-ligand 1; TC, tumor cell.

FIG. 9 shows pathological regression in the modified Intent-to-treat population. Data cut-off of Nov. 10, 2022 (N=740). Pathological regression is summarized based on patients with evaluable % RVT. The waterfall plots show pathological regression of the primary tumor in the D arm (left panel) and PBO arm (right panel). The black stars indicate patients with evidence of carcinoma present in any examined lymph nodes or whose lymph nodes are not evaluable. pCR was defined as a lack of any viable tumor cells after complete evaluation of the resected lung cancer specimen and all sampled regional lymph nodes. MPR was defined as ≤10% viable tumor cells in lung primary tumor after complete evaluation of the resected lung cancer specimen. D, durvalumab; MPR, major pathological response; PBO, placebo; pCR, pathological complete response; RVT, residual viable tumor.

FIG. 10 shows the most frequently reported adverse events (AEs) * of the phase 3 study (safety analysis set). DCO=Nov. 10, 2022. *Displayed are AEs reported with a frequency of ≥10% in the D arm during the overall study period; the overall study period spans from the first dose of study Tx (D/PBO/CT) until the earliest of: the last dose of study Tx or surgery+90 days (taking the latest dose of D/PBO/CT/date of surgery, +90 days); the DCO date; or the date of the first dose of subsequent anti-cancer Tx. †Two patients (n=1 per arm) had decreased appetite with an outcome of death (grade 5); the fatal event in the D arm was assessed as possibly related to study Tx by the investigator. ‡Six patients had grade 5 COVID-19 events (D arm, n=5; PBO arm, n=1); all COVID-19 deaths were assessed by the investigator as unrelated to study Tx (note: COVID-19 is summarized as a grouped term comprising the ‘COVID-19’ and ‘COVID-19 pneumonia’ preferred terms).

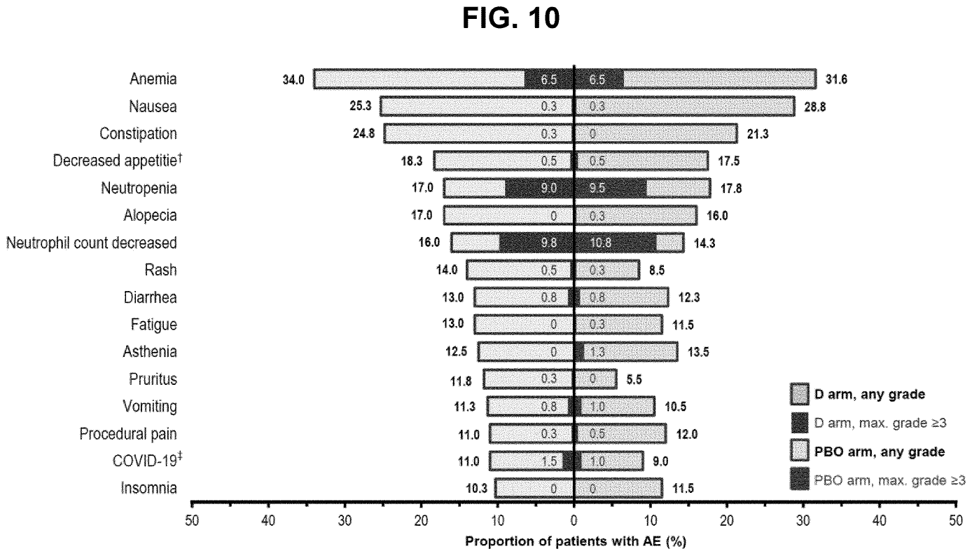

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiments of the present disclosure.

### DETAILED DESCRIPTION OF THE DISCLOSURE

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

As used herein, “one or more” can refer to a selection from a group.

Percentages disclosed herein can vary in amount by +10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. The term “about” also includes the exact amount. For example, “about 5%” means “about 5%” and also “5%.” The term “about” can also refer to ±10% of a given value or range of values. Therefore, about 5% also means 4.5%-5.5%, for example. Additionally, “about” or “comprising essentially of” can mean a range of up to ±10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition. Unless otherwise clear from context, all numerical values provided herein are modified by the term “about.”

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the terms “or” and “and/or” can describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

The present disclosure generally relates to methods for treating resectable non-small cell lung cancer patients (R-NSCLC). This disclosure demonstrates that perioperative durvalumab plus neoadjuvant chemotherapy results in significant improvement of both pathological complete response and event free survival among patients with resectable NSCLC.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

In an aspect, this disclosure provides for methods of treating a patient identified as having resectable non-small cell lung carcinoma (R-NSCLC) comprising administering to the patient a combination therapy comprising durvalumab and a platinum-based chemotherapy.

In an aspect, this disclosure provides a method of treating a patient identified as having resectable non-small cell lung carcinoma (R-NSCLC), the method comprising administering to the patient about 1500 mg of durvalumab and a platinum-based chemotherapy about every 3 weeks (Q3W) for about 4 cycles; removing the R-NSCLC by surgery; and then administering to the patient about 1500 mg of durvalumab about every four weeks (Q4W) for at least about 12 weeks.

In an aspect, this disclosure provides, a combination therapy comprising durvalumab and a platinum-based chemotherapy for use in treating resectable non-small cell lung cancer (R-NSCLC) in a patient in need thereof.

In an aspect, this disclosure provides, a combination therapy for use in treating a patient identified as having resectable non-small cell lung cancer (R-NSCLC), wherein the combination therapy comprises: administering to the patient about 1500 mg of durvalumab and a platinum-based chemotherapy about every 3 weeks (Q3W) for about 4 cycles; and then administering to the patient about 1500 mg of durvalumab about every four weeks (Q4W) for at least about 12 weeks after removal of the R-NSCLC by surgery.

In an aspect, this disclosure provides, a combination therapy formulated for use in treating a patient identified as having resectable non-small cell lung cancer (R-NSCLC), wherein the combination therapy comprises: administering to the patient about 1500 mg of durvalumab and a platinum-based chemotherapy about every 3 weeks (Q3W) for about 4 cycles; and then administering to the patient about 1500 mg of durvalumab about every four weeks (Q4W) for at least about 12 weeks after removal of the R-NSCLC by surgery.

In an aspect, this disclosure provides, the use of a combination therapy comprising durvalumab and a platinum-based chemotherapy for the manufacture of a medicament for treating resectable non-small cell lung cancer (R-NSCLC) in a patient in need thereof.

In an aspect, this disclosure provides, the use of a durvalumab and a platinum-based chemotherapy in the manufacture of a medicament for treating resectable non-small cell lung cancer (R-NSCLC) in a combination therapy, wherein the combination therapy comprises: administering to the patient about 1500 mg of durvalumab and a platinum-based chemotherapy about every 3 weeks (Q3W) for about 4 cycles; removing the R-NSCLC by surgery; and then administering to the patient about 1500 mg of durvalumab about every four weeks (Q4W) for at least about 12 weeks.

As used herein, the terms “treat,” “treatment,” or “treating” when used in the context of treating cancer refer to reducing disease pathology, reducing or eliminating disease symptoms, promoting increased survival rates, and/or reducing discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

As used herein, the terms “subject,” “individual,” or “patient,” refer to any subject, particularly a mammalian subject, for whom diagnosis, prognosis, or therapy is desired. Mammalian subjects include, for example, humans, non-human primates, dogs, cats, guinea pigs, rabbits, rats, mice, horses, cattle, bears, and so on. The term “patient” may refer to a human.

In an aspect, this disclosure provides methods of treating a patient identified as having resectable non-small cell lung cancer (R-NSCLC). In some embodiments, the patient having R-NSCLC is newly diagnosed. In some embodiments, the patient having R-NSCLC is previously untreated. In some embodiments, the patient having R-NSCLC is histologically or cytologically documented as having stage II or stage III cancer (according to the American Joint Committee on Cancer [AJCC] Cancer Staging Manual, version 8). In some embodiments, the patient having R-NSCLC is histologically or cytologically documented as having stage IIA to select [N2] stage IIIB cancer (according to the American Joint Committee on Cancer [AJCC] Cancer Staging Manual, version 8). In some embodiments, the patient having R-NSCLC is newly diagnosed, previously untreated, and histologically or cytologically documented, resectable NSCLC (stage IIA to select [N2] stage IIIB according to the American Joint Committee on Cancer [AJCC] Cancer Staging Manual, version 8). In some embodiments, the patient having R-NSCLC is has resectable Stage IIA to select [ie, N2]Stage IIIB) disease (according to Version 8 of the IASLC Staging Manual in Thoracic Oncology 2016). In some embodiments, the patient having R-NSCLC is a candidate for lobectomy, sleeve resection, or bilobectomy as planned surgery at the time of enrollment.

In some embodiments, the PD-L1 status of the patient's R-NSCLC is determined prior to treatment. In some embodiments, the R-NSCLC of the patient has a PD-L1 tumor cell expression that is <1% (that is, fewer than 1% of the tumor cells express PD-L1). In some embodiments, the R-NSCLC of the patient has a PD-L1 tumor cell expression that is 1-49%. In some embodiments, the R-NSCLC of the patient has a PD-L1 tumor cell expression that is ≥50%. In certain embodiments, a patient's tumor PD-L1 expression status is determined using Ventana PD-L1 (SP263) immunohistochemistry (IHC) assay applied to formalin-fixed, paraffin-embedded tissue sample(s).

In some embodiments, the R-NSCLC is a squamous cell carcinoma. In some embodiments, the R-NSCLC is a non-squamous cell carcinoma. In some embodiments, the patient's tumor EGFR and ALK status of the R-NSCLC is determined prior to treatment. In some embodiments, the patient does not have an EGFR mutation and/or an ALK translocation. In some embodiments, patients with Kirsten rat sarcoma (KRAS) mutations in their tumors were not required to be tested for EGFR/ALK and patients with squamous cell carcinoma were not required to be tested for ALK.

As used herein, the term “durvalumab” refers to an antibody that selectively binds PD-L1 and blocks the binding of PD-L1 to the PD-1 and CD80 receptors. The durvalumab antibody is disclosed in U.S. Pat. No. 9,493,565 (referred to as “2.14H90PT”), which is incorporated by reference herein in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (“ADCC”). In certain embodiments, the triple mutation refers to the IgG1 Fc region comprising a L234F/L235E/P331S triple mutation (EU numbering; see also U.S. Pat. No. 9,493,565). Durvalumab can relieve PD-L1-mediated suppression of human T-cell activation in vitro and inhibits tumor growth in a xenograft model via a T-cell dependent mechanism. Amino acid sequences of Durvalumab are as follows:

Durvalumab sequences

MEDI4736 VL
EIVLTQSPGTLSLSPGERATLSCRASQRVSSSYLAWYQQKPGQAPRLL
IYDASSRATGIPDRFSGSGSGTDFTLTISRLEPEDFAVYYCQQYGSLP
WTFGQGTKVEIK (SEQ ID NO. 1)
MEDI4736 VL CDR1
RASQRVSSSYLA (SEQ ID NO. 2)
MEDI4736 VL CDR2
DASSRAT (SEQ ID NO. 3)
MEDI4736 VL CDR3
QQYGSLPWT (SEQ ID NO. 4)
MEDI4736 VH
EVQLVESGGGLVQPGGSLRLSCAASGFTFSRYWMSWVRQAPGKGLEWV
ANIKQDGSEKYYVDSVKGRFTISRDNAKNSLYLQMNSLRAEDTAVYYC
AREGGWFGELAFDYWGQGTLVTVSS (SEQ ID NO. 5)
MEDI4736 VH CDR1
GFTFSRYWMS (SEQ ID NO. 6)
MEDI4736 VH CDR2 (SEQ ID NO. 7)
NIKQDGSEKYYVDSVKG
MEDI4736 VH CDR3
EGGWFGELAFDY (SEQ ID NO. 8)

As used herein, the term “platinum-based chemotherapy” refers to chemotherapy drugs that contain the element platinum. Platinum-based chemotherapy has been used to treat many different types of cancer. In some embodiments, a platinum-based chemotherapy refers to chemotherapy treatment comprising comprises at least one of one or more of carboplatin, cisplatin, nedaplatin, and oxaliplatin. In some embodiments, a platinum-based chemotherapy can also refer to chemotherapy treatment comprising at least one or more of triplatin tetranitrate, phenanthriplatin, picoplatin, and satraplatin. In certain embodiments, a platinum-based chemotherapy further comprises one or more of afatinib, cetuximab, bevacizumab, erlotinib, gemcitabine, paclitaxel, pemetrexed, and vemurafenib. In certain embodiments, a platinum-based chemotherapy comprises carboplatin and paclitaxel. In certain embodiments, a platinum-based chemotherapy comprises cisplatin and gemcitabine. In certain embodiments, a platinum-based chemotherapy comprises carboplatin and gemcitabine. In certain embodiments, a platinum-based chemotherapy comprises carboplatin and pemetrexed. In certain embodiments, a platinum-based chemotherapy comprises cisplatin and pemetrexed. In certain embodiments, a platinum-based chemotherapy comprises a carboplatin area under the serum drug concentration-time curve (AUC) dose of about 5 to 6 mg/mL/min. In certain embodiments, a platinum-based chemotherapy comprises a cisplatin dose of about 75 mg/m². In certain embodiments, a platinum-based chemotherapy comprises a paclitaxel dose of about 200 mg/m². In certain embodiments, a platinum-based chemotherapy comprises a pemetrexed dose of about 500 mg/m². In certain embodiments, a platinum-based chemotherapy comprises a gemcitabine dose of about 1250 mg/m².

In some embodiments, the combination therapy may be neoadjuvant durvalumab and neoadjuvant chemotherapy. In some embodiments, the combination therapy may be perioperative durvalumab and neoadjuvant chemotherapy. In some embodiments, the combination therapy may comprise neoadjuvant durvalumab, neoadjuvant chemotherapy and adjuvant durvalumab.

In some embodiments, the combination therapy comprises administering about 1000 mg to about 2000 mg durvalumab once about every 14 to 28 days for up to about 3 to 6 cycles prior to surgery to remove the resectable NSCLC and then about 1000 mg to about 2000 mg durvalumab once about every 14 to 28 days for up to about 12 cycles following the surgery. In some embodiments, the combination therapy comprises administering about 1500 mg durvalumab once about every three weeks (Q3W) for up to a maximum of about 4 cycles prior to surgery to remove the resectable NSCLC and then about 1500 mg durvalumab once about every four weeks (Q4W) for about 12 cycles following the surgery. In some embodiments, the combination therapy comprises administering an amount of durvalumab that is determined from the patient's weight. For example, if a patient's weight falls to 30 kg or below [≤30 kg], the patient should receive weight-based dosing equivalent to about 20 mg/kg of durvalumab [or placebo] about Q3W or about Q4W until the weight improves to >30 kg, at which point the patient should start receiving the fixed dosing of durvalumab about 1500 mg [or placebo] about Q3W or about Q4W.

In some embodiments, durvalumab can be provided as a 500-mg vial solution for infusion after dilution. In some embodiments, the solution contains 50 mg/mL durvalumab, 26 mM histidine/histidine-hydrochloride, 275 mM trehalose dihydrate, and 0.02% w/v polysorbate 80; it has a pH of 6.0 and density of 1.054 g/mL. In certain embodiments, a dose of about 1500 mg (for patients >30 kg in weight) can be administered using an IV bag containing 0.9% (w/v) saline or 5% (w/v) dextrose, with a final durvalumab concentration ranging from 1 to 15 mg/mL and delivered through an IV administration set with a 0.2- or 0.22-μm filter. In some embodiments, the amount of durvalumab is determined from the patient's weight. For example, if a patient's weight falls to ≤30 kg, weight-based dosing at 20 mg/kg can be administered using an IV bag selected such that the final concentration is within 1 to 15 mg/mL. In some embodiments, a standard infusion time is 1 hour (±10 mins); however, if there are interruptions, the total allowed time must not exceed 8 hours at room temperature.

In an aspect, this disclosure provides methods of treating a patient identified as having resectable non-small cell lung carcinoma (R-NSCLC), comprising (i) administering to the patient about 1000-2000 mg of durvalumab and a platinum-based chemotherapy about every 14-28 days for about 3 to 6 cycles; (ii) removing the R-NSCLC by surgery; and then (iii) administering to the patient about 1000-2000 mg of durvalumab about every 14 to 28 days for at least about 12 weeks.

In certain embodiments, after the surgery to remove the resectable NSCLC, patients should start durvalumab administration as soon as clinically feasible and within about 10 weeks from the surgery to remove the resectable NSCLC. A minimum of about 3 weeks is recommended between surgery to remove the resectable NSCLC and the start of durvalumab treatment (first post-surgical scan must be performed prior to starting adjuvant treatment and post-operative radiotherapy, if required). Complete post-operative wound healing must have occurred following any surgery.

In some embodiments, surgery to remove the resectable NSCLC should happen within about 40 days from the last administration of durvalumab.

In some embodiments, surgery to remove the resectable NSCLC comprises pneumonectomy, lobectomy, segmentectomy or wedge resection, sleeve resection, or bilobectomy. Pneumonectomy is surgery that removes an entire lung, and might be needed if the tumor is close to the center of the chest. Lobectomy is the removal of one or more of the affected 5 lobes (3 on the right lung and 2 on the left lung) of the lungs, and the entire lobe(s) containing the tumor(s) is removed. Segmentectomy or wedge resection removes only part of a lobe (i.e. a segment or a wedge), and might be used if a patient does not have enough normal lung function to withstand removing the whole lobe. Sleeve resection involves removeing a portion of the bronchus, in addition to the surrounding lung lobe, and then rejoin the healthy ends of the bronchus together. In some embodiments, surgery to remove the resectable NSCLC comprises anatomic pulmonary resection of the NSCLC. In some embodiments, either open thoracotomy or video-assisted thoracoscopic surgery access can be carried out as appropriate to the expertise of the surgeon. In some embodiments, lung-sparing anatomic resection (sleeve lobectomy) is preferred over pneumonectomy, if anatomically appropriate and margin-negative resection is achieved. In some embodiments, T3 (invasion) and T4 local extension tumors may require en-bloc resection of the involved structure with negative margins.

In some embodiments, patients can receive post-operative radiotherapy (PORT) following surgery to remove the resectable NSCLC. Post-operative radiotherapy (PORT) can be given within about 8 weeks after surgery. In some embodiments, adjuvant durvalumab starts no longer than about 3 weeks after the end of PORT. In some embodiments, adjuvant durvalumab starts no more than about 10 weeks after surgery. In some embodiments, post-operative radiotherapy (PORT) is allowed for patients in which it is indicated according to local guidance. In some embodiments, PORT can include, but is not limited to, doses ranging from 50 to 60 Gy, 1.8 to 2 Gy per fraction, 5 fractions a week; for patients with positive margins of disease (RI). In some embodiments, PORT can include, but is not limited to, doses ranging from 60 to 66 Gy, 1.8 to 2 Gy per fraction, 5 fractions a week. In some embodiments, intensity-modulated radiation therapy (IMRT) or 3D-conformal radiotherapy (3D-CRT) is allowed.

In some embodiments, the success of the method of treatment is determined by an improvement in pathological complete response (pCR) as compared to standard of care.

In some embodiments, the success of the method of treatment is determined by an improvement in major pathological response (mPR) as compared to standard of care.

In some embodiments, the success of the method of treatment is determined by an improvement event free survival (EFS) as compared to standard of care, e.g., neoadjuvant chemotherapy, such as neoadjuvant platinum-based chemotherapy.

Thus success of treatment may be defined as an increase in EFS, pCR, DFS, OS, and/or mPR in patients treated with (i) neoadjuvant durvalumab and neoadjuvant chemotherapy compared to patients treated with neoadjuvant chemotherapy only; or (ii) perioperative durvalumab and neoadjuvant chemotherapy compared to patients treated with neoadjuvant chemotherapy only; or (iii) neoadjuvant durvalumab, neoadjuvant chemotherapy and adjuvant durvalumab compared to patients treated with neoadjuvant chemotherapy only.

In some embodiments, the success of the method of treatment is determined by an improvement in pathological complete response (pCR) by at least about 13% as compared to standard of care. In some embodiments, the success of the method of treatment is determined by an improvement in major pathological response (mPR) by at least about 21% as compared to standard of care. In some embodiments, the success of the method of treatment is determined by an improvement in event-free survival (EFS) of at least about 13 months as compared to standard of care.

In certain embodiments, the use of the combination therapy results in improvement in one or more of EFS, pCR, mPR, DFS, and OS compared to a standard of care. In certain embodiments, the use of the combination therapy results in improvement in one or more of EFS, pCR, and mPR, DFS, and OS compared to a platinum-based chemotherapy, or platinum-based chemotherapy following or preceding surgery.

As used herein, the term “Standard of care” (SoC) can refer to one or more of:
1) Carboplatin+paclitaxel: carboplatin AUC 6 (mg/mL/min) and paclitaxel 200 mg/m² via IV infusion on Day 1 of each 3-week cycle, for 4 cycles (for squamous tumor histology).
2) Cisplatin+gemcitabine: cisplatin 75 mg/m² via IV infusion on Day 1 of each 3-week cycle, for 4 cycles, and gemcitabine 1250 mg/m² via IV infusion on Day 1 and Day 8 of each 3-week cycle, for 4 cycles (for squamous tumor histology). In the event of unfavorable tolerability, patients can switch from cisplatin to carboplatin therapy at any point during the study (assuming eligibility for the switched therapy is met). In patients with comorbidities or unable to tolerate cisplatin per Investigators judgment, carboplatin AUC 5 (mg/mL/min) can be administered from cycle 1.
3) Pemetrexed+cisplatin: pemetrexed 500 mg/m² and cisplatin 75 mg/m² via IV infusion on Day 1 of each 3-week cycle, for 4 cycles (for non-squamous tumor histology). In the event of unfavorable tolerability, patients can switch from cisplatin to carboplatin therapy at any point during the study (assuming eligibility for the switched therapy is met). In patients with comorbidities or unable to tolerate cisplatin per Investigators judgment, carboplatin AUC 5 (mg/mL/min) can be administered from cycle 1.
4) Pemetrexed+carboplatin: pemetrexed 500 mg/m² and carboplatin AUC 5 (mg/m/min) via IV infusion on Day 1 of each 3-week cycle, for 4 cycles (for non-squamous tumor histology).

As used herein, the terms “event-free survival” or “EFS” refer to the time from randomization to the first of the following: a) local or distant recurrence as determined by BICR using RECIST 1.1 assessments; b) death due to any cause (event date is the date of death); c) PD that precludes surgery (event date is the date of this determination) or PD discovered and reported by the Investigator upon attempting surgery that prevents completion of surgery (event date is the date of the first attempt at surgery). Event-free survival can be analyzed using the log-rank test based on blinded independent central review (BICR) assessment using RECIST v1.1 and pathology review stratified by disease stage (Stage II versus Stage III) and by PD-L1 expression status (<1% versus >1%) on the mITT. The p-value can be obtained from the stratified log-rank test using the Efron (Hertz-Picciotto and Rockhill “Validity and efficiency of approximation methods for tied survival times in Cox regression.” Biometrics 1997; 53(3):1151-6.) approach for handling ties.

As used herein, the terms “pathological complete response” or “pathological CR” or “pCR” refer to the proportion of patients who have 0% residual viable tumor cells within all resected tissue (including primary lung lesion and lymph nodes) following neoadjuvant treatment as assessed by central pathology laboratory. Patients who are not evaluable per central pathology assessment (this includes patients with R2 margins) or who do not have a surgical specimen can be considered as non-pCR (e.g., pathology assessments captured as “non-evaluable” or “missing,” as appropriate). Central pathology assessment of pCR will be performed according to the recommended methods and definitions described by IASLC 2020 (Travis et al. “IASLC Multidisciplinary Recommendations for Pathologic Assessment of Lung Cancer Resection Specimens After Neoadjuvant Therapy.” J Thorac Oncol. 2020 May; 15(5):709-740. doi:10.1016/j.jtho.2020.01.005). The analysis can be performed using a Cochran-Mantel-Haenszel (CMH) test, stratified by disease stage (Stage II versus Stage III) and PD-L1 expression status (<1% versus ≥1%). The effect of treatment will be estimated by the difference in proportions between treatment groups, together with their corresponding CI and p-value.

As used herein, the terms “disease-free survival” and “DFS” refer to the time from the date of surgery until the first date of disease recurrence as determined by BICR using RECIST 1.1 assessments (local or distant), or date of death due to any cause, whichever occurs first. Pathological confirmation from biopsied lesions, can also be taken into consideration (as applicable). A new primary malignancy, confirmed by pathology, is not considered a DFS event. Disease-free survival can be analyzed using the log-rank test based on BICR assessment using RECIST 1.1 and pathology review stratified by disease stage (Stage II versus Stage III) and by PD-L1 expression status (<1% versus ≥1%) on the modified resected analysis set. The p-value can be obtained from the stratified log-rank test using the Efron (Hertz-Picciotto and Rockhill 1997) approach for handling ties.

As used herein, the terms “major pathological response” and “mPR” refer to the proportion of patients with 10% or less residual viable tumor tissue in lung primary tumor after neoadjuvant treatment at the time of resection as assessed per central pathology laboratory. Patients who are not evaluable per central pathology assessment (including patients with R2 margins) or who do not have a surgical specimen can be considered as having non-mPR (e.g., response captured as “non-evaluable” or “missing,” as appropriate). The analysis can be performed using a CMH test, stratified by disease stage (Stage II versus Stage III) and PDL1 expression status (<1% versus ≥1%). The effect of treatment can be estimated by the difference in proportions between treatment groups, together with their corresponding CI and p-value.

As used herein, the terms “overall survival” and “OS” refer to the time from the date of randomization until death due to any cause regardless of whether the subject withdraws from randomized therapy or receives another anticancer therapy. Any patient not known to have died at the time of analysis will be censored based on the last recorded date on which the patient was known to be alive. Overall survival can be analyzed in the mITT population using the same methodology as described for EFS. The effect of treatment can be estimated by the HR together with its corresponding CI. Kaplan-Meier plots can be presented by treatment arm. Analyses can be performed using Kaplan-Meier estimates of OS to estimate the number of OS patients at 12, 24, 36, 48, and 60 months.

Intent-to-treat (ITT) population set: The ITT analysis set includes all randomized patients. Treatment groups are compared on the basis of randomized study treatment, regardless of the treatment actually received. Patients who were randomized but did not subsequently go on to receive study treatment are included in the analysis in the treatment group to which they were randomized.

Modified Intent-to-treat (mITT) population set: The mITT includes all patients in the ITT, excluding those whose tumors have EGFRm/ALK translocation. Unless otherwise specified, the mITT is used for all efficacy analyses, including PROs. Treatment arms are compared based on randomized study treatment, regardless of the treatment actually received.

Resected population set: The resected set can consist of all patients in the ITT who had surgical resection following the neoadjuvant period, who do not have R2 margins, and whose first scan following surgery shows no evaluable disease (defined as no post-surgery R2 margins and no RECIST evidence of disease).

Modified Resected population set: The modified resected set can consist of all patients in the resected set, excluding those whose tumors have EGFRm/ALK translocation. Unless otherwise specified, this analysis set is used for DFS only. Treatment arms are compared based on randomized study treatment, regardless of the treatment actually received.

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Without limiting the disclosure, a number of embodiments of the disclosure are described below for purpose of illustration.

### EXAMPLES

The Examples that follow are illustrative of specific embodiments of the disclosure, and various uses thereof. They are set forth for explanatory purposes only and should not be construed as limiting the scope of the invention in any way.

### Example 1: Neoadjuvant/Adjuvant Durvalumab for the Treatment of Patients with Resectable Non-Small Cell Lung Cancer (R-NSCLC)

The AEGEAN (NCT03800134) study described herein is a phase III, double-blind, placebo-controlled, multi-center international study of assessed perioperative (i.e. neoadjuvant and adjuvant) durvalumab plus neoadjuvant chemotherapy for the treatment of patients with resectable stage II and stage III non-small cell lung cancer (R-NSCLC).

While the primary treatment for early-stage non-small cell lung cancer (NSCLC [Stages I to IIIA]) is curative surgery, the 5-year survival rate for patients treated with surgery alone remains low, ranging from 67% (Stage IA) to 23% (Stage IIIA) (Mountain 1997). Early studies of immunotherapy in the neoadjuvant setting in patients with resectable NSCLC have shown promising clinical activity as well as acceptable safety profiles (Forde et al. “Neoadjuvant PD-1 blockade in resectable lung cancer.” N Engl J Med 2018; 378:1976-86; Shu et al. “Neoadjuvant atezolizumab+chemotherapy in patients with resectable non-small cell lung cancer (NSCLC).” Poster presented at: ASCO Annual Meeting 2018.). It may be possible that combining chemotherapy with durvalumab prior to surgery can have clinical benefit over chemotherapy+placebo. Therefore, in this Phase III study, the administration of durvalumab+a platinum-based chemotherapy prior to surgery, followed by further administration of durvalumab after surgery, was investigated to determine if this therapy regimen can improve activity and therefore long-term clinical outcomes for patients with resectable NSCLC.

### Patients

The study enrolled approximately 1300 patients and randomized approximately 800 eligible patients. Eligible patients had newly diagnosed, previously untreated, histologically or cytologically documented, resectable NSCLC (stage IIA to select [N2] stage IIIB according to the American Joint Committee on Cancer [AJCC] Cancer Staging Manual, version 8). Patients were randomized in a 1:1 ratio to receive either durvalumab plus a platinum-based chemotherapy before surgery followed by durvalumab post-surgery (Arm 1; a platinum-based chemotherapy plus durvalumab intravenously every three weeks (four cycles) before surgery, followed by durvalumab intravenously every four weeks (12 cycles)) or placebo plus a platinum-based chemotherapy before surgery followed by placebo post-surgery (Arm 2; a platinum-based chemotherapy plus placebo intravenously every three weeks (four cycles) before surgery, followed by placebo intravenously every four weeks (12 cycles)). Patients were also stratified by disease stage (Stage II versus Stage III) and by PD-L1 expression status (<1% versus ≥1%).

Patients had resectable (Stage IIA to select [ie, N2]Stage IIIB) disease (according to Version 8 of the IASLC Staging Manual in Thoracic Oncology 2016) and were candidates for lobectomy, sleeve resection, or bilobectomy as planned surgery at the time of enrollment. At screening, complete surgical resection of the primary NSCLC was deemed achievable by performing a multidisciplinary evaluation, which included a thoracic surgeon who performs lung cancer surgery as a prominent part of his/her practice. Additional inclusion criteria necessitated an age of 18 years or older; an Eastern Cooperative Oncology Group performance status of 0 or 1; an estimated life expectancy of ≥12 weeks; documented tumor PD-L1 status (as assessed at a central laboratory using the VENTANA SP263 immunohistochemistry assay); and >1 lesion, not previously irradiated, that qualified as a Response Evaluation Criteria in Solid Tumors version 1.1 (RECIST v1.1) target lesion.

T4 tumors were only eligible if they were defined as T4 based only on their size (more than 7 cm); any other reason for T4 (e.g., adherent to any of the following structures: diaphragm, mediastinum, heart, great vessels, trachea, recurrent laryngeal nerve, esophagus, vertebral body, carina) were considered ineligible.

Nodal status was investigated with whole body 18F-fluoro-deoxyglucose positron emission tomography (FDG-PET), plus contrast-enhanced computed tomography (CT). If positron emission tomography (PET)/CT scan was positive in the mediastinum, or if scan was negative, but there was T>3 cm, central tumor, or clinical N1 (cN1), then it was recommended that nodal status be proven by biopsy via endobronchial ultrasound, mediastinoscopy, or thoracoscopy. Mandatory brain magnetic resonance imaging was performed (MRI; preferred) with IV contrast or brain CT with IV contrast at the time of staging.

Patients had no prior exposure to immune-mediated therapy including, but not limited to, other anti-CTLA-4, anti-PD-1, anti-PD-L1, and anti-PD-L2 antibodies, excluding therapeutic anticancer vaccines.

Confirmation of a patient's tumor PD-L1 status occurred prior to randomization using Ventana PD-L1 (SP263) immunohistochemistry (IHC) assay applied to formalin fixed paraffin embedded tissue sample with testing completed by the central laboratory.

Confirmation of EGFR and ALK status was also performed during screening. Patients with Kirsten rat sarcoma (KRAS) mutations in their tumors were not required to be tested for EGFR/ALK and patients with squamous cell carcinoma were not required to be tested for ALK. Patients with documented EGFR/ALK aberrations were excluded from efficacy analyses in the modified intent-to-treat population (N=740)

Patients were considered suitable for inclusion if the planned surgery to be performed was lobectomy, sleeve resection, or bilobectomy, as determined by the attending surgeon based on the baseline findings. The patient was deemed to have adequate cardiac and lung function, according to a multidisciplinary assessment. A pre- or post-bronchodilator FEV1 of 1.0 L and >40% postoperative predicted value. Use of these cut-off values to assess candidacy for resection was guided by the results of cardiopulmonary exercise testing as outlined in the European Society for Medical Oncology (ESMO) guidelines on pretreatment risk assessment. Both an FEV1 and a DLCO test were required for assessing lung function prior to resection.

TABLE 1
Baseline Characteristics and Planned Treatment
in the Modified Intent-to-Treat Population.*

| | | Durvalumab arm | | Placebo arm | |
|---|---|---|---|---|---|
| Characteristics^{†} | | (N = 366) | | (N = 374) | |
| Age | Median (range), years | 65.0 | (30-88) | 65.0 | (39-85) |
| | ≥75 years, n (%) | 44 | (12.0) | 36 | (9.6) |
| Sex, n (%) | Male | 252 | (68.9) | 278 | (74.3) |
| | Female | 114 | (31.1) | 96 | (25.7) |
| ECOG PS, n (%) | 0 | 251 | (68.6) | 255 | (68.2) |
| | 1 | 115 | (31.4) | 119 | (31.8) |
| Race^{‡}, n (%) | Asian | 143 | (39.1) | 164 | (43.9) |
| | White | 206 | (56.3) | 191 | (51.1) |
| | Other | 17 | (4.6) | 19 | (5.1) |
| Region, n (%) | Asia | 142 | (38.8) | 163 | (43.6) |
| | Europe | 141 | (38.5) | 140 | (37.4) |
| | North America | 43 | (11.7) | 43 | (11.5) |
| | South America | 40 | (10.9) | 28 | (7.5) |
| Smoking status, n (%) | Current | 95 | (26.0) | 95 | (25.4) |
| | Former | 220 | (60.1) | 223 | (59.6) |
| | Never | 51 | (13.9) | 56 | (15.0) |
| Disease stage | II | 104 | (28.4) | 110 | (29.4) |
| (AJCC 8^{th} edition), n (%) | IIIA | 173 | (47.3) | 165 | (44.1) |
| | IIIB | 88 | (24.0) | 98 | (26.2) |
| TNM classification^{¶}: | T1 | 44 | (12.0) | 43 | (11.5) |
| primary tumor, n (%) | T2 | 97 | (26.5) | 108 | (28.9) |
| | T3 | 128 | (35.0) | 129 | (34.5) |
| | T4 | 97 | (26.5) | 94 | (25.1) |
| TNM classification^{¶}: | N0 | 110 | (30.1) | 102 | (27.3) |
| regional lymph nodes, n (%) | N1 | 75 | (20.5) | 87 | (23.3) |
| | N2 | 181 | (49.5) | 185 | (49.5) |
| | Single-station | 141 | (38.5) | 132 | (35.3) |
| | Multi-station | 34 | (9.3) | 40 | (10.7) |
| Histology, n (%) | Squamous | 169 | (46.2) | 191 | (51.1) |
| | Non-squamous | 196 | (53.6) | 179 | (47.9) |
| PD-L1 expression, n (%) | TC <1% | 122 | (33.3) | 125 | (33.4) |
| | TC 1-49% | 135 | (36.9) | 142 | (38.0) |
| | TC ≥50% | 109 | (29.8) | 107 | (28.6) |
| Planned neoadjuvant | Cisplatin | 100 | (27.3) | 96 | (25.7) |
| platinum agent, n (%) | Carboplatin | 266 | (72.7) | 278 | (74.3) |

Data cut-off of Nov. 10, 2022 (N = 740).
*The modified intent-to-treat population included all patients who were randomized, excluding patients with documented EGFR/ALK aberrations.
^{†}Characteristics with missing or other responses were histology (0.3% in the durvalumab arm and 1.1% in the placebo arm had ‘other’ histology), disease stage (0.3% in the durvalumab arm had stage IV disease and 0.3% in the placebo arm had stage III [NOS] disease, as reported per the eCRF), and N2 lymph node station status (1.6% in the durvalumab arm and 3.5% in the placebo arm had N2 disease with missing data on single-station versus multi-station classification).
^{‡}Race was self-reported per the eCRF.
^{¶}All patients were M0 except one patient in the durvalumab arm who was classified as M1 (NOS).
AJCC, American Joint Committee on Cancer; ECOG PS, Eastern Cooperative Oncology Group performance status; eCRF, electronic case report form; NOS, not otherwise specified; PD-L1, programmed cell death-ligand 1; TC, tumor cell; TNM, tumor, [lymph] nodes, metastasis.

TABLE 2
Representativeness of Study Participants.

| | AEGEAN | |
|---|---|---|
| | Study | |
| | mITT, | |
| Demographics | N = 740 | Observations |
| Age, median (range) | 65.0 (30-88) | Patients with resectable NSCLC are on average |
| Age, mean + SD | 64.0 ± 8.8 | approximately 65 years of age across |
| | | geographies. The age of patients on the |
| | | AEGEAN study was representative of a real- |
| | | world population. |
| Sex | | Globally NSCLC affects more men than women. The |
| Male | 71.6% | gender balance in the AEGEAN study was |
| Female | 28.4% | representative of a real-world population. |
| ECOG performance | | The majority of patients diagnosed with resectable |
| status | | NSCLC have good performance status (ECOG |
| 0 | 68.4% | performance status of 0), a trend that was also |
| 1 | 31.6% | borne out in the AEGEAN study. |
| ≥2 | 0% | |
| Unknown/Missing | 0% | |
| Smoking Status | | Lung cancer is strongly associated with smoking, with |
| Current smoker/Former | 86% | approximately 80% of patients having a history |
| smoker | | of smoking. This was also reflected in the |
| Never smoker | 24% | AEGEAN study population. |
| Region | | The AEGEAN study enrolled patients across 28 |
| Asia | 41% | countries and 3 continents. The race and |
| Europe | 38% | ethnicity composition of the AEGEAN study |
| North America | 12% | are indicative of a large, diverse, and global |
| South America | 9% | clinical trial. |
| Race | | |
| Asian | 2.0% | |
| White | 79.6% | |
| Black/African | 9.6% | |
| American | | |
| Other/Unknown | 9.3% | |
| Ethnicity | | |
| Hispanic/Latino | 2.5% | |
| Not Hispanic/Latino | 87.5% | |
| Pre-planned Platinum | | In real-world practice, many patients may have |
| Backbone | | contraindications to cisplatin-based |
| Carboplatin | 77.3% | chemotherapy. The AEGEAN study allowed for |
| Cisplatin | 27.8% | flexibility in the choice of platinum agent, and |
| | | as such is more reflective of a real-world |
| | | treatment regimen. |

ECOG, Eastern Cooperative Oncology Group; mITT, modified intent-to-treat; SD, standard deviation.

TABLE 3
Patient Disposition and Treatment Summary in
the Modified Intent-to-treat Population.*

| | | Durvalumab arm | | Placebo arm | |
|---|---|---|---|---|---|
| Study phase^{†} | | (N = 366) | | (N = 374) | |
| Neoadjuvant | Randomized, n (%) | 366 | (100) | 374 | (100) |
| phase | Received treatment, n (%) | 366 | (100) | 371 | (99.2) |
| | Completed 4 cycles of both | 310 | (84.7) | 326 | (87.2) |
| | chemotherapy agents, n (%) | | | | |
| | Completed 4 cycles of | 318 | (86.9) | 331 | (88.5) |
| | durvalumab/placebo, n (%) | | | | |
| Surgery | Underwent surgery^{‡}, n (%) | 295 | (80.6) | 302 | (80.7) |
| | Did not undergo surgery^{‡¶}, n (%) | 71 | (19.4) | 72 | (19.3) |
| | Completed surgery^{‡}, n (%) | 284 | (77.6) | 287 | (76.7) |
| | R0 resection, n (% of patients who | 269 | (94.7) | 262 | (91.3) |
| | completed surgery) | | | | |
| | Did not complete surgery^{‡}, n (%) | 11 | (3.0) | 15 | (4.0) |
| Adjuvant | Started durvalumab/placebo^{§}, n (%) | 241 | (65.8) | 237 | (63.4) |
| phase | Completed durvalumab/placebo, n (%) | 88 | (24.0) | 79 | (21.1) |
| (ongoing) | Discontinued durvalumab/placebo, n (%) | 68 | (18.6) | 70 | (18.7) |
| | Ongoing durvalumab/placebo, n (%) | 85 | (23.2) | 88 | (23.5) |

Data cut-off of Nov. 10, 2022 (N = 740).
*The modified intent-to-treat population included all patients who were randomized, excluding patients with documented EGFR/ALK aberrations.
^{†}Except where specified otherwise, percentages were calculated using the full modified intent-to-treat population as the denominator.
^{‡}As per the investigator's assessment. Patients who ‘underwent’ surgery were those for whom curative-intent thoracic surgery was attempted regardless of whether it was completed. Patients who ‘completed’ surgery were those for whom curative-intent thoracic surgery was completed (assessed by the investigator at the time of surgery).
^{¶}Includes patients who had surgery outside of the study.
^{§}For patients to be eligible for adjuvant durvalumab or placebo, surgery must have been completed with R0/R1 resection margins and no evidence of disease on post-surgical RECIST assessment. RECIST, Response Evaluation Criteria in Solid Tumors.

TABLE 4
Baseline Characteristics in the Intent-to-treat Population

| | | Durvalumab arm | | Placebo arm | |
|---|---|---|---|---|---|
| Characteristics* | | (N = 400) | | (N = 402) | |
| Age | Median (range), years | 65.0 | (30-88) | 65.0 | (39-85) |
| | ≥75 years, n (%) | 48 | (12.0) | 38 | (9.5) |
| Sex, n (%) | Male | 262 | (65.5) | 291 | (72.4) |
| | Female | 138 | (34.5) | 111 | (27.6) |
| ECOG PS, n (%) | 0 | 278 | (69.5) | 277 | (68.9) |
| | 1 | 122 | (30.5) | 125 | (31.1) |
| Race^{†}, n (%) | Asian | 165 | (41.3) | 187 | (46.5) |
| | White | 216 | (54.0) | 196 | (48.8) |
| | Other | 19 | (4.8) | 19 | (4.7) |
| Region, n (%) | Asia | 164 | (41.0) | 186 | (46.3) |
| | Europe | 147 | (36.8) | 144 | (35.8) |
| | North America | 50 | (12.5) | 44 | (10.9) |
| | South America | 39 | (9.8) | 28 | (7.0) |
| Smoking status, | Current | 96 | (24.0) | 97 | (24.1) |
| n (%) | Former | 232 | (58.0) | 231 | (57.5) |
| | Never | 72 | (18.0) | 74 | (18.4) |
| Disease stage | II | 119 | (29.8) | 120 | (29.9) |
| (AJCC 8^{th} edition), | IIIA | 186 | (46.5) | 178 | (44.3) |
| n (%) | IIIB | 94 | (23.5) | 103 | (25.6) |
| TNM classification^{‡}: | T1 | 53 | (13.3) | 48 | (11.9) |
| primary tumor, n (%) | T2 | 108 | (27.0) | 119 | (29.6) |
| | T3 | 141 | (35.3) | 136 | (33.8) |
| | T4 | 98 | (24.5) | 99 | (24.6) |
| TNM classification^{‡}: | N0 | 118 | (29.5) | 110 | (27.4) |
| regional lymph | N1 | 83 | (20.8) | 94 | (23.4) |
| nodes, n (%) | N2 | 199 | (49.8) | 198 | (49.3) |
| | Single-station | 151 | (37.8) | 140 | (34.8) |
| | Multi-station | 38 | (9.5) | 45 | (11.2) |
| Histology, n (%) | Squamous | 173 | (43.3) | 226 | (56.5) |
| | Non-squamous | 192 | (47.8) | 206 | (51.2) |
| PD-L1 expression, | TC <1% | 133 | (33.3) | 134 | (33.3) |
| n (%) | TC 1-49% | 151 | (37.8) | 158 | (39.3) |
| | TC ≥50% | 38 | (9.5) | 110 | (27.4) |
| EGFR mutation | Detected | 26 | (6.5) | 25 | (6.2) |
| status, n (%) | Not detected | 327 | (81.8) | 328 | (81.6) |
| | Not tested due to KRAS | 1 | (0.3) | 1 | (0.2) |
| | Not tested due to squamous | 43 | (10.8) | 46 | (11.4) |
| | histology | | | | |
| | Unknown | 3 | (0.8) | 2 | (0.5) |
| ALK translocation | Detected | 8 | (2.0) | 3 | (0.7) |
| status, n (%) | Not detected | 298 | (74.5) | 296 | (73.6) |
| | Not tested due to squamous | 84 | (21.0) | 98 | (24.4) |
| | histology | | | | |
| | Unknown | 10 | (2.5) | 5 | (1.2) |

Data cut-off of Nov. 10, 2022 (N = 802).
*Characteristics with missing or other responses were histology (0.3% in the durvalumab arm and 1.0% in the placebo arm had ‘other’ histology), disease stage (0.3% in the durvalumab arm had stage IV disease and 0.2% in the placebo arm had stage III [NOS] disease, as reported per the eCRF), and N2 lymph node station status (2.5% in the durvalumab arm and 3.2% in the placebo arm had N2 disease with missing data on single-station vs. multi-station classification).
^{†}Race was self-reported per the eCRF.
^{‡}All patients were M0 except one patient in the durvalumab arm who was classified as M1 (NOS).
AJCC, American Joint Committee on Cancer; ECOG PS, Eastern Cooperative Oncology Group performance status; eCRF, electronic case report form; NOS, not otherwise specified; PD-L1, programmed cell death-ligand 1; TC, tumor cell; TNM, tumor, [lymph] nodes, metastasis.

TABLE 5
Summary of Adverse Events in the Safety Analysis Set.*

| Overall study period | | | | |
|---|---|---|---|---|
| (inclusive of the neoadjuvant, surgical, | Durvalumab arm | | Placebo arm | |
| and adjuvant treatment phases)^{†} | (N = 400) | | (N = 399) | |
| Any-grade all-causality adverse events, n (%) | 386 | (96.5) | 378 | (94.7) |
| Maximum grade 3 or 4 | 169 | (42.3) | 173 | (43.4) |
| Serious adverse events | 150 | (37.5) | 126 | (31.6) |
| Outcome of death | 23 | (5.8) | 15 | (3.8) |
| Leading to discontinuation of durvalumab/placebo | 48 | (12.0) | 24 | (6.0) |
| Leading to cancellation of surgery | 7 | (1.8) | 4 | (1.0) |
| Any-grade adverse events possibly related to | 346 | (86.5) | 322 | (80.7) |
| durvalumab/placebo/chemotherapy, n (%) | | | | |
| Maximum grade 3 or 4 | 129 | (32.3) | 132 | (33.1) |
| Outcome of death^{‡} | 7 | (1.8) | 2 | (0.5) |

Data cut-off of Nov. 10, 2022 (N = 799).
*The safety analysis set includes all randomized patients who received ≥1 dose of study treatment; adverse events were graded using Common Terminology Criteria for Adverse Events version 5.0.
^{†}The overall study period spans from the first dose of study treatment (durvalumab/placebo/chemotherapy) until the earliest of: the last dose of study treatment or surgery + 90 days (taking the latest dose of durvalumab or placebo or chemotherapy or the date of surgery, +90 days); the data cut-off date; or the date of the first dose of subsequent anti-cancer treatment.
^{‡}Adverse events with an outcome of death included events assessed by the investigator as possibly related to any study treatment and include interstitial lung disease (n = 2) and immune-mediated lung disease, pneumonitis, hemoptysis, myocarditis, and decreased appetite (n = 1 each) in the durvalumab arm and pneumonia and infection (n = 1 each) in the placebo arm.

### Study Design and Treatment

Approximately 800 patients with resectable NSCLC (Stage IIA to select Stage IIIB; either squamous or non-squamous) were randomized in a 1:1 ratio to receive either durvalumab plus platinum-based chemotherapy before surgery followed by durvalumab post-surgery (Arm 1) or placebo plus platinum-based chemotherapy before surgery followed by placebo post-surgery (Arm 2). Patients were stratified by disease stage (Stage II versus Stage III) and by programmed cell death ligand-1 (PD-L1) expression status (<1% versus ≥1%).

Patients received 4 cycles of durvalumab or placebo plus a platinum-based chemotherapy (every 3 weeks [Q3W]) followed by surgery to remove the NSCLC. Surgery consisted of lobectomy, sleeve resection, or bilobectomy, as determined by the attending surgeon based on the baseline findings. Patients whose planned surgery at enrollment includes pneumonectomy, segmentectomies, or wedge resections at eligibility assessment were not eligible for this study. All patients were staged and managed according to the National Comprehensive Cancer Network 2020 Guidelines (version 1.2021). Surgery was expected within 40 days from the dose.

Patients started durvalumab or placebo administration following surgery as soon as clinically feasible and within 10 weeks from surgery (except for patients receiving post-operative radiation therapy, which were started within 8 weeks after surgery; durvalumab/placebo was given within 3 weeks from the end of post-operative radiation therapy). Patients randomized to receive durvalumab plus platinum-based chemotherapy prior to surgery received an additional 12 cycles of durvalumab 1500 mg every 4 weeks (Q4W), while patients randomized to receive placebo plus platinum-based chemotherapy prior to surgery received an additional 12 cycles of placebo Q4W.

### Treatments and Treatment Duration:

Patients received 1500 mg durvalumab or placebo via intravenous (IV) infusion Q3W for up to a maximum of 4 cycles prior to surgery and Q4W for 12 cycles following surgery, unless there was unacceptable toxicity, withdrawal of consent, or another discontinuation criterion was met. If a patient's weight (WT) falls to 30 kg or below (≤30 kg), then the patient received WT-based dosing equivalent to 20 mg/kg of durvalumab [or placebo]Q3W or Q4W after consultation between Investigator and Study Physician/Medical Scientist, until the WT improved to >30 kg, at which point the patient started receiving the fixed dosing of durvalumab 1500 mg [or placebo]Q3W or Q4W).

### Standard of Care (SoC):

Patients received one of the following four SoC regimens, based on the tumor histology and Investigator's discretion, as part of their treatment regimen prior to surgery. Options for patients with squamous histology were carboplatin plus paclitaxel or cisplatin plus gemcitabine (or carboplatin plus gemcitabine for patients who had comorbidities or who are unable to tolerate cisplatin per the investigator's judgment). Options for non-squamous histology were pemetrexed plus either cisplatin or carboplatin.
1) Squamous tumor histology: Carboplatin+paclitaxel: carboplatin area under the serum drug concentration-time curve (AUC) 6 (mg/mL/min) and paclitaxel 200 mg/m² via IV infusion on Day 1 of each 3-week cycle, for 4 cycles.
2) Squamous tumor histology: Cisplatin+gemcitabine: cisplatin 75 mg/m² via IV infusion on Day 1 of each 3-week cycle, for 4 cycles, and gemcitabine 1250 mg/m² via IV infusion on Day 1 and Day 8 of each 3-week cycle, for 4 cycles. In the event of unfavorable tolerability, patients switched from cisplatin to carboplatin therapy at any point during the study (assuming eligibility for the switched therapy was met). In patients with comorbidities or unable to tolerate cisplatin per Investigators judgment, carboplatin AUC 5 (mg/mL/min) was administered from cycle 1.
3) Non-squamous tumor histology: Pemetrexed+cisplatin: pemetrexed 500 mg/m² and cisplatin 75 mg/m² via IV infusion on Day 1 of each 3-week cycle, for 4 cycles. In the event of unfavorable tolerability, patients switched from cisplatin to carboplatin therapy at any point during the study (assuming eligibility for the switched therapy was met). In patients with comorbidities or unable to tolerate cisplatin per Investigators judgment, carboplatin AUC 5 (mg/mL/min) was administered from cycle 1.
4) Non-squamous tumor histology: Pemetrexed+carboplatin: pemetrexed 500 mg/m² and carboplatin AUC 5 (mg/mL/min) via IV infusion on Day 1 of each 3-week cycle, for 4 cycles.

Duration of treatment: Unless there is unacceptable toxicity, withdrawal of consent, or another discontinuation criterion was met, treatment with 4 cycles of durvalumab or placebo plus platinum-based chemotherapy (Q3W) was administered prior to surgery, and treatment with durvalumab or placebo continued for 12 cycles (Q4W) following surgery (±1 cycle of platinum-based chemotherapy).

Surgery was expected within 40 days of the last IP dose prior to surgery, and patients started durvalumab or placebo administration following surgery as soon as clinically feasible and within 10 weeks from surgery (except for patients receiving post-operative radiation therapy, which was started within 8 weeks after surgery; durvalumab/placebo was given within 3 weeks from the end of post-operative radiation therapy). For patients to be eligible for adjuvant durvalumab or placebo, surgery must have been completed with R0 or R1 resection margins with no evidence of disease on the first post-surgical assessment using RECIST v1.1.

Progression during treatment: Treatment was stopped at the time of local or distant recurrence determined by investigator using RECIST 1.1 or clinical progression, including progression that precludes surgery or progression discovered upon attempting surgery.

Follow-up of patients post discontinuation of study drug: Patients who discontinued study treatment due to toxicity or symptomatic deterioration, clinical progression (excluding progression that precludes surgery or progression discovered upon attempting surgery), or who commenced subsequent anticancer therapy in the absence of local or distant recurrence (for resected patients) or RECIST 1.1-defined radiological progression of disease (PD) (for patients who did not have surgery for reasons other than PD) were followed up with tumor assessments until local or distant recurrence (for resected patients) or RECIST 1.1-defined radiological PD (for patients who did not have surgery for reasons other than PD) as determined by investigator and followed up for survival. These patients were not eligible for retreatment at any time.

### Endpoints and Assessments

The two primary efficacy endpoints in this study were event-free survival (EFS; per blinded independent central review [BICR]) and pathological complete response (pCR; evaluated centrally). Other secondary endpoints included evaluation of the primary endpoints and key secondary endpoints in patients with PD-L1 expression≥1%; pharmacokinetics and immunogenicity; patient-reported outcomes; and safety. In accordance with FDA guidance on clinical trial endpoints for the approval of cancer drugs and biologics (FDA 2018), EFS was defined as the time from randomization to any of the following events: progression of disease (PD) that precludes surgery, local or distant recurrence, or death due to any cause. More specifically, EFS was defined as the time from randomization to the earliest of: (1) progressive disease that precludes surgery; (2) progressive disease discovered and reported by the investigator upon attempting surgery that prevents completion of surgery; (3) local or distant recurrence using blinded independent central review according to RECIST v1.1; or (4) death from any cause. Failure to undergo/complete surgery for reasons other than progressive disease was not considered an EFS event (i.e. patients remained in follow-up for RECIST-defined progression).

Primary tumors and sampled lymph nodes were assessed for pathological response to neoadjuvant treatment by central pathology review using recommendations from the IASLC. To be eligible for pathological assessment, patients should have had three cycles of neoadjuvant study treatment per the protocol; patients who were not eligible for assessment (including those with R2 resection margins by local assessment) or who did not have a surgical specimen were classified as non-responders. pCR was defined as a lack of any viable tumor cells after complete evaluation of the resected lung cancer specimen and all sampled regional lymph nodes, and MPR was defined as ≤10% of viable tumor cells in the lung primary tumor (Travis et al. “IASLC Multidisciplinary Recommendations for Pathologic Assessment of Lung Cancer Resection Specimens After Neoadjuvant Therapy.” J Thorac Oncol. 2020 May; 15(5):709-740. doi: 10.1016/j.jtho.2020.01.005; Hellmann et al. “Pathological response after neoadjuvant chemotherapy in resectable non-small-cell lung cancers: proposal for the use of major pathological response as a surrogate endpoint.” Lancet Oncol 2014; 15(1):e42-50; Cottrell et al. “Pathologic features of response to neoadjuvant anti-PD-1 in resected non-small-cell lung carcinoma: a proposal for quantitative immune-related pathologic response criteria (irPRC).” Ann Oncol 2018; 29(8):1853-1860).

While time-to-event endpoints and OS are generally considered to be the preferred outcome measure for Phase III cancer studies in early disease, they often result in NSCLC studies that are very long. The time-to-event endpoints and OS are therefore endpoints that are difficult to achieve within a reasonable time period. Endpoints that demonstrate early clinical benefit, such as mPR and pCR, may also be used to evaluate efficacy in early-stage NSCLC studies, provided that the magnitude of benefit is clinically meaningful.

Secondary efficacy endpoints in this study included disease-free survival (DFS) (modified resected set and PD-L1 TC≥1% resected set), pCR (PD-L1 TC≥1% analysis set), major pathological response (mPR) (mITT and PD-L1 TC≥1% analysis set), EFS (PD-L1 TC≥1% analysis set), and overall survival (OS) (mITT and PD-L1 TC≥1% analysis set).

Disease-free survival (DFS) was defined as the time from the date of surgery until the first date of disease recurrence (local or distant), or date of death due to any cause, whichever occurred first. Pathological confirmation from biopsied lesions, if performed according to Investigators judgment and local practice, was also taken into consideration (as applicable). A new primary malignancy confirmed by pathology was not considered a DFS event.

TABLE 6
Endpoints and Assessments

| Primary objective: | Endpoint/variable: |
|---|---|
| To compare the efficacy of durvalumab + chemotherapy | EFS |
| administered prior to surgery followed by durvalumab | |
| post-surgery compared with placebo + chemotherapy | |
| administered prior to surgery followed by placebo post- | |
| surgery in terms of EFS | |
| To compare the activity of durvalumab + chemotherapy | pCR (lack of any viable tumor cells |
| administered prior to surgery compared with placebo + | after complete evaluation in the resected |
| chemotherapy administered prior to surgery in terms of | lung cancer specimen and all sampled |
| pCR | regional lymph nodes) |

| | Endpoints/variables: |
|---|---|
| Secondary objectives: | |
| To compare the efficacy of durvalumab + chemotherapy | DFS |
| administered prior to surgery followed by durvalumab | |
| post-surgery compared with placebo + chemotherapy | |
| administered prior to surgery, followed by placebo post- | |
| surgery in terms of DFS | |
| To compare the activity of durvalumab + chemotherapy | mPR (≤10% viable tumor cells in lung |
| administered prior to surgery compared with placebo + | primary tumor after complete evaluation |
| chemotherapy administered prior to surgery in terms of | in the resected lung cancer specimen) |
| mPR | |
| To compare the efficacy of durvalumab + chemotherapy | OS |
| administered prior to surgery followed by durvalumab | |
| post-surgery compared with placebo + chemotherapy | |
| administered prior to surgery followed by placebo post- | |
| surgery in terms of OS | |
| To compare the efficacy of durvalumab + chemotherapy | EFS |
| administered prior to surgery followed by durvalumab | pCR |
| post-surgery compared with placebo + chemotherapy | DFS |
| administered prior to surgery followed by placebo post- | mPR |
| surgery in patients with PD-L1 TC ≥ 1% in terms of EFS, | OS |
| pCR, DFS, mPR, and OS | |
| To compare disease-related symptoms and HRQOL in | Change from baseline in EORTC QLQ- |
| patients treated with durvalumab + chemotherapy | C30 and EORTC QLQ-LC13 and time |
| administered prior to surgery followed by durvalumab | to deterioration in EORTC QLQ-C30. |
| post-surgery compared with placebo + chemotherapy | |
| administered prior to surgery followed by placebo post- | |
| surgery | |
| To assess the PK of durvalumab | Concentration of durvalumab |
| To investigate the immunogenicity of durvalumab | Presence of ADAs for durvalumab |
| Safety objective: | |
| To compare the safety and tolerability profile of | AEs, physical examinations, vital signs |
| durvalumab + chemotherapy administered prior to | (including BP, pulse, and ECGs), and |
| surgery followed by durvalumab post-surgery compared | laboratory findings (including clinical |
| with placebo + chemotherapy administered prior to | chemistry, hematology, and urinalysis) |
| surgery followed by placebo post-surgery | |

ADA Anti-drug antibody;
AE Adverse event;
BP Blood pressure;
DFS Disease-free survival;
ECG Electrocardiogram;
EFS Event-free survival;
EORTC European Organisation for Research and Treatment of Cancer;
HRQoL Healthrelated quality of life;
IHC Immunohistochemistry;
mPR Major pathological response;
OS Overall survival;
pCR Pathological complete response;
PD-L1 Programmed cell death ligand-1;
PD-L1 TC ≥ 1% Expression of PD-L1 on tumor membrane, at any intensity, in ≥1% of tumor cells;
PK Pharmacokinetic(s);
QLQ-C30 30-item Core Quality of Life Questionnaire;
QLQ-LC13 13-item Lung Cancer Quality of Life Questionnaire

Tumors were evaluated per RECIST v1.1 using imaging collected at the following timepoints: baseline (≤28 days before randomization); after completing neoadjuvant treatment and before surgery; 5 weeks (±2 weeks) after surgery and before starting adjuvant treatment; every 12 weeks (±1 week) until Week 48 after surgery; every 24 weeks (±2 weeks) until Week 192 (i.e., ~4 years post-surgery); and every 48 weeks (±2 weeks) thereafter until local or distant recurrence, consent withdrawal, or death.

Safety was monitored throughout the study and adverse events (AEs) were graded using the National Cancer Institute Common Toxicity Criteria for AEs version 5.0.

Efficacy assessments were performed and determined by pathology review or a BICR using RECIST 1.1 assessment, as applicable, including EFS (mITT and PD-L1 TC≥1% analysis set), pCR (mITT and PD-L1 TC≥1% analysis set), mPR (mITT and PD-L1 TC≥1% analysis set), DFS (modified resected set and PD-L1 TC≥1% resected set), and OS (mITT and PD-L1 TC≥1% analysis set). The EFS event of PD that precludes surgery or PD discovered during surgery that prevents completion of surgery will be determined by the Investigator.

At the first planned interim analysis the EFS follow-up in censored patients was 11.7 months (range: 0.0-46.1).

### Statistical Analysis

The two primary endpoints in this study were event-free survival (EFS) and pathological complete response (pCR) in the modified intent-to-treat (mITT) population. The key secondary endpoints were disease-free survival (DFS) in the modified resected population and major pathological response (mPR) and overall survival (OS) in the mITT population. The family-wise error rate was strongly controlled at a 5% 2-sided level via multiple testing procedure. The testing procedure was hierarchical in that it starts with testing the 2 primary endpoints EFS and pCR. The overall 2-sided 5% type I error was split between the 2 primary endpoints EFS and pCR. An alpha level of 4.5% was allocated to the EFS analysis, and an alpha level of 0.5% was allocated to the pCR analysis.

The mITT population included all randomized patients, excluding those whose tumors had epidermal growth factor receptor mutation (EGFRm) or anaplastic lymphoma kinase (ALK) translocation. Unless otherwise specified, the mITT was used for all efficacy analyses, including PROs. Treatment groups were compared on the basis of randomized study treatment, regardless of the treatment actually received.

A pCR interim analysis (IA) was performed when about 400 mITT patients had the opportunity to undergo surgery (actual N=402), and final analysis was performed once all mITT patients (actual N=740) had the opportunity to undergo surgery. pCR and MPR rates were compared between the study arms using a stratified CMH test. CIs for the difference between arms were estimated using MN confidence limits. The first interim EFS analysis (presented here) was planned at ~30% maturity (actual EFS maturity: 31.9%).

Comparisons between the study arms were analyzed using stratified log-rank tests. HRs and 95% CIs were estimated from stratified Cox PH models. EFS medians and landmarks were estimated using the KM method.

For pathological endpoints, response rates were compared between treatment arms using a stratified Cochran-Mantel-Haenszel test; the treatment effect was estimated by the differences in response rates, with their corresponding 95% confidence intervals (CIs) calculated by the stratified Miettinen and Nurminen method. EFS was compared between the treatment arms using a stratified log-rank test; the treatment effect was estimated by HRs and 95% CIs calculated with stratified Cox-proportional-hazards models. Medians and landmark rates for EFS were estimated using the Kaplan-Meier method. Stratification for the primary and key secondary endpoints was by disease stage and PD-L1 expression. Planned analyses of the primary endpoints in prespecified subgroups were performed. For pCR, the differences in response rates were calculated for each subgroup, with corresponding 95% CIs estimated using an unstratified Miettinen and Nurminen method; for EFS, HRs and 95% CIs were calculated for each subgroup using a Cox-proportional-hazards model with treatment as the only covariate.

To strongly control the type I error at 5% (two-sided), a hierarchical multiple testing procedure with gatekeeping strategy was used across the primary endpoints and alpha-controlled secondary endpoints. Initially, 0.5% alpha and 4.5% alpha were allocated to pCR and EFS, respectively. The alpha was split between the interim and final analyses (IA and FA, respectively) using the Lan-DeMets spending function that approximates an O'Brien Fleming approach to account for multiple time point assessments (Lan and DeMets, “Discrete Sequential Boundaries for Clinical Trials”, Biometrika Vol. 70, No. 3 (December, 1983), pp. 659-663). Positivity for pCR enabled alpha recycling to the key secondary endpoint MPR, which in turn could be recycled to EFS (to provide a total 5% alpha). Based on a total of 0.5% alpha allocated to the pCR endpoint, the planned IA of pCR (assuming 400 patients in the modified intent-to-treat [mITT]population at the IA, 740 patients in the mITT population at the FA) had 55% power to detect a statistically significant between-arm difference of 12% with a two-sided significance level of 0.0078%; MPR (an alpha-controlled secondary endpoint) was also formally analyzed at the IA. For EFS, a 33-month nonlinear (k=2) accrual was assumed with a 3-month delay in hazard, whereby the assumed hazard ratio for the first 3 months was 1.0 and a hazard ratio (HR) of 0.63 was assumed after 3 months to give an approximate overall HR of 0.67 at the time of the FA. Based on a total of 4.5% alpha allocated to the EFS endpoint, for the first interim analysis of EFS, if the true overall HR is 0.69, with 224 EFS events (per blinded independent central review) in the mITT population (N=740) the study would provide 50% power to demonstrate a statistically significant EFS effect with a two-sided significance level of 0.6649%. Since pCR and MPR were statistically significant, EFS was tested with a total 5% alpha allocated. The actual significance level was calculated based on the observed number of patients or events at the interim analysis compared with the planned number of patients or events at the final analysis for each endpoint, respectively.

TABLE 7
Interim Anlaysis cohort.

| | | | PBO | |
|---|---|---|---|---|
| Population | Definition | D arm | arm | Total |
| ITT | All randomized patients | 400 | 402 | 802 |
| mITT | ITT excluding patients with | 366 | 374 | 740 |
| | documented EGFR/ALK | | | |
| | aberrations | | | |
| pCR IA cohort^{†} | First ~400 patients in the mITT | 196 | 206 | 402 |
| Safety | ITT patients who received ≥1 | 400 | 399 | 799 |
| analysis set | dose of study Tx | | | |

*A hierarchical testing procedure was employed for the primary and key secondary efficacy endpoints.
^{†}The pCR IA cohort was a subset of the mITT population used for efficacy analyses at the pCR IA.
CMH, Cochran-Mantel-Haenszel; D, durvalumab; KM, Kaplan-Meier; MN, Miettinen and Nurminen: PBO, placebo: PH, proportional hazards.

### Results

### Patients and Treatments

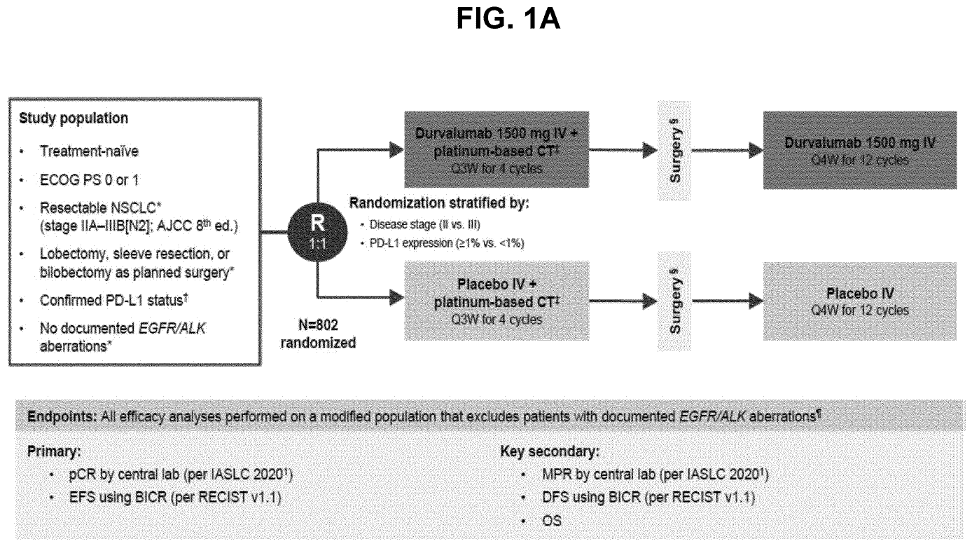

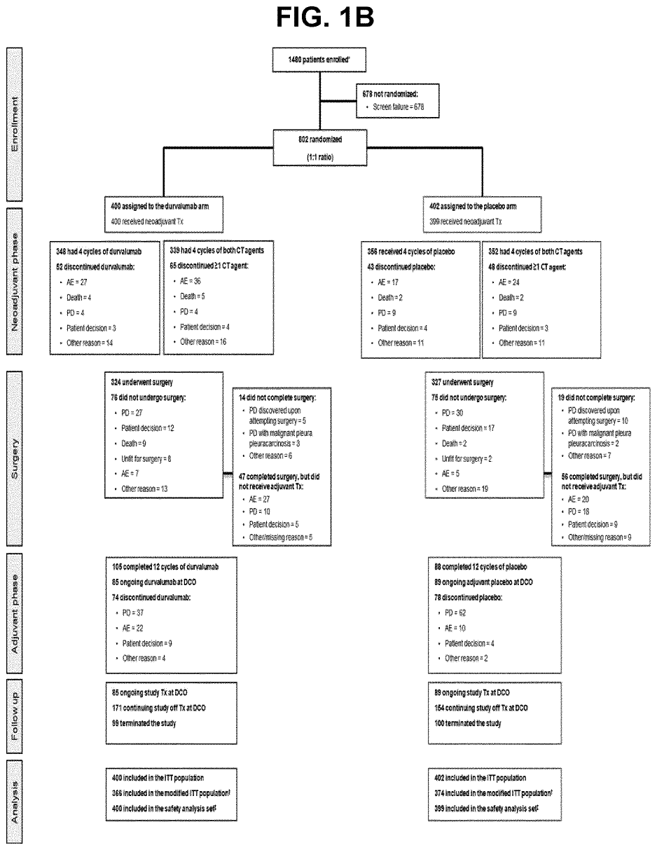

1,480 patients were enrolled of whom 802 patients were randomized to either the durvalumab arm (n=400) or placebo arm (n=402), comprising the ITT population (FIG. 1B). The characteristics of the ITT population (Table 4) were generally representative of a real-world population of patients with resectable NSCLC (Table 2). The mITT population (excluding patients with known EGFR/ALK aberrations) comprised 740 patients, with 366 and 374 randomized to the durvalumab and placebo arms, respectively. Baseline demographics and clinical characteristics and planned neoadjuvant chemotherapy doublet regimens were largely balanced between the treatment arms in the mITT population (Table 1). Median age was 65.0 years, and most patients were male (71.6%), had a performance status of 0 (68.4%), and were current or former smokers (85.5%). Over 70% of patients had stage III disease, and half of all patients had N2 disease. Approximately equal proportions of patients had squamous and non-squamous histology. Across both treatment arms, 33.4% of patients had tumor PD-L1 expression of <1%, and carboplatin was the planned neoadjuvant platinum agent for over 70% of patients.

At the data cut-off for this first planned interim analysis of EFS, median follow-up for EFS in censored patients was 11.7 months (range, 0.0 to 46.1). In the mITT population, approximately 8500 of patients had completed four cycles of both chemotherapy agents in each treatment arm, and over 60% of all patients had started adjuvant durvalumab or placebo (Table 3; see Table 8 for details of neoadjuvant treatment exposure). Few patients in either treatment arm (5.8% of the mITT population) received post-operative radiotherapy (as permitted by the protocol, if indicated, according to the discretion of the treating physician). At the data cut-off for this analysis, 24.0% and 21.10% of patients in the mITT population had completed 12 cycles of adjuvant durvalumab or placebo, respectively; 18.6% and 18.7% of patients in each arm had prematurely discontinued adjuvant study treatment, respectively. Approximately a quarter of all patients in the mITT population were still receiving adjuvant study treatment (23.2% and 23.5% of patients in the durvalumab and placebo arms, respectively).

TABLE 8
Neoadjuvant Treatment Exposure in the Safety Analysis Set.

| | Durvalumab arm | | Placebo arm | |
|---|---|---|---|---|
| | (N = 400) | | (N = 399) | |
| Patients receiving neoadjuvant treatment, n (%) | 400 | (100) | 399 | (100) |
| Type of SoC chemotherapy, n (%) | | | | |
| Cisplatin + Gemcitabine | 46 | (11.5) | 43 | (10.8) |
| Cisplatin + Paclitaxel | 0 | | 1 | (0.3) |
| Cisplatin + Pemetrexed | 63 | (15.8) | 60 | (15.0) |
| Carboplatin + Gemcitabine | 11 | (2.8) | 9 | (2.3) |
| Carboplatin + Paclitaxel | 122 | (30.5) | 140 | (35.1) |
| Carboplatin + Pemetrexed | 158 | (39.5) | 146 | (36.6) |
| Total duration of treatment (weeks): | | | | |
| Any SoC chemotherapy, median (range) | 12.1 | (2.0-20.7) | 12.1 | (3.0-22.7) |
| Durvalumab/placebo, median (range) | 12.1 | (2.0-19.0) | 12.1 | (3.0-22.7) |
| Cycles of any SoC chemotherapy, n (%) | | | | |
| ≥4 cycles | 347 | (86.8) | 356 | (89.2) |
| ≥3 cycles | 382 | (95.5) | 375 | (94.0) |
| ≥2 cycles | 390 | (97.5) | 387 | (97.0) |
| ≥1 cycle | 400 | (100) | 399 | (100) |
| Cycles of durvalumab/placebo received, n (%) | | | | |
| ≥4 cycles | 349 | (87.3) | 358 | (89.7) |
| ≥3 cycles | 386 | (96.5) | 377 | (94.5) |
| ≥2 cycles | 392 | (98.0) | 389 | (97.5) |
| ≥1 cycle | 400 | (100) | 399 | (100) |

Data cut-off of Nov. 10, 2022 (N = 799). The safety analysis set includes all randomized patients who received ≥1 dose of study treatment. SoC, standard of care.

### Surgery Summary

At the data cut-off, approximately 81% of patients in each treatment arm (Table 3; mITT population) had undergone surgery (noting that curative-intent thoracic surgery was attempted regardless of whether it was completed). In total, 77.6% of patients in the durvalumab arm and 76.7% of patients in the placebo arm had completed surgery (i.e. curative-intent thoracic surgery deemed completed as assessed by the investigator), among whom a slightly higher proportion had R0 resection in the durvalumab arm versus the placebo arm (94.7% vs. 91.3%). (See Table 9 and Table 10 for a summary of the most common reasons for not undergoing or completing surgery in the ITT population, details of surgical delays in the safety analysis set [Table 9] and details of surgery and surgical outcomes in the mITT population [Table 10].)

TABLE 9
Details of Surgical Delays in the Safety Analysis Set.

| | Durvalumab arm | | Placebo arm | |
|---|---|---|---|---|
| Surgical detail | (N = 400) | | (N = 399) | |
| Patients with delayed surgery, n (%) | 57 | (14.3) | 68 | (17.0) |
| Reason for surgical delay, n (%)* | | | | |
| Unresolved toxicity from | 5 | (1.3) | 4 | (1.0) |
| neoadjuvant Tx | | | | |
| Durvalumab/Placebo | 2 | (0.5) | 2 | (0.5) |
| SoC | 3 | (0.8) | 2 | (0.5) |
| Logistical reasons | 30 | (7.5) | 38 | (9.5) |
| Adverse event | 11 | (2.8) | 13 | (3.3) |
| Other | 14 | (3.5) | 13 | (3.3) |
| Length of delay in surgery, n (%) | | | | |
| <2 weeks | 31 | (7.8) | 39 | (9.8) |
| 2 to <4 weeks | 14 | (3.5) | 22 | (5.5) |
| 4 to <6 weeks | 7 | (1.8) | 3 | (0.8) |
| ≥6 weeks | 5 | (1.3) | 4 | (1.0) |

Data cut-off of Nov. 10, 2022 (N = 799). The safety analysis set includes all randomized
patients who received ≥1 dose of study treatment.
*Reasons for surgical delay are not mutually exclusive for patients with multiple reasons per delay or subjects with multiple delays (although a patient can only be counted once per category).

TABLE 10
Details of Surgery and Surgical Outcomes in
the Modified Intent-to-treat Population.

| | Durvalumab arm | | Placebo arm | |
|---|---|---|---|---|
| Surgical detail | (N = 366) | | (N = 374) | |
| Patients who underwent | 295 | (80.6) | 302 | (80.7) |
| surgery, n (%) | | | | |
| Patients who completed | 284 | (77.6) | 287 | (76.7) |
| surgery, n (%) | | | | |
| Margins, n (%)* | | | | |
| R0 | 269 | (94.7) | 262 | (91.3) |
| R1 | 12 | (4.2) | 22 | (7.7) |
| R2 | 2 | (0.7) | 2 | (0.7) |
| Missing | 1 | (0.4) | 1 | (0.3) |
| Surgery procedure | | | | |
| performed, n (%) | | | | |
| Lobectomy | 238 | (65.0) | 221 | (59.1) |
| Sleeve resection | 7 | (1.9) | 14 | (3.7) |
| Bilobectomy | 13 | (3.6) | 20 | (5.3) |
| Pneumonectomy | 27 | (7.4) | 29 | (7.8) |
| Sleeve resection (bronchial) | 2 | (0.5) | 2 | (0.5) |
| Sleeve resection (arterial) | 0 | | 1 | (0.3) |
| Wedge resection | 1 | (0.3) | 2 | (0.5) |
| Other | 7 | (1.9) | 13 | (3.5) |
| Surgical approach, n (%) | | | | |
| Open Procedure | 145 | (39.6) | 153 | (40.9) |
| Minimally invasive | 145 | (39.6) | 142 | (38.0) |
| Other | 4 | (1.1) | 6 | (1.6) |
| Missing | 1 | (0.3) | 1 | (0.3) |
| Days from last neoadjuvant Tx | 34.0 | (12-91) | 34.0 | (13-103) |
| dose to surgery, median (range) | | | | |
| Days from surgery to first dose | 50.0 | (22-136) | 52.0 | (21-141) |
| of adjuvant Tx, median (range) | | | | |

Data cut-off of Nov. 10, 2022 (N = 740).
*For the summary of resection margin status only, percentages are calculated using the number of patients who completed surgery as the denominator.

### Efficacy

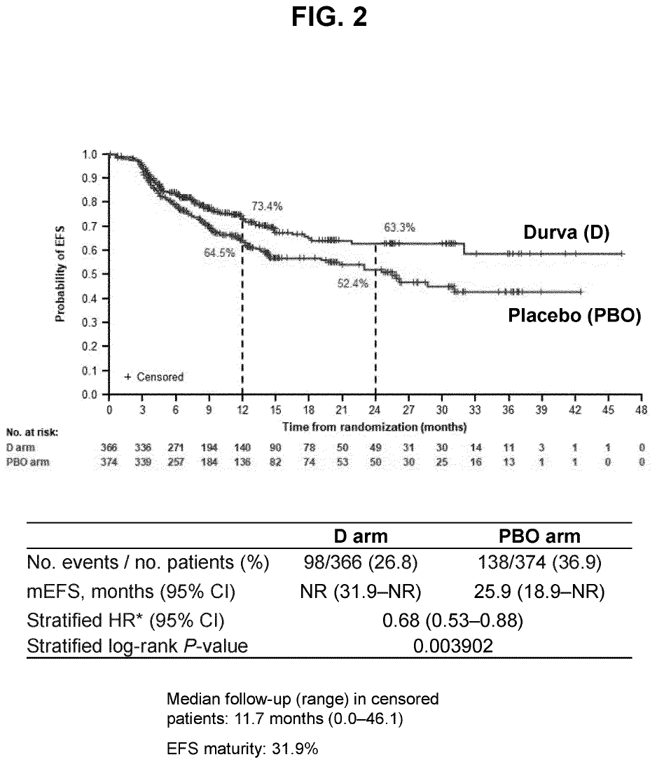

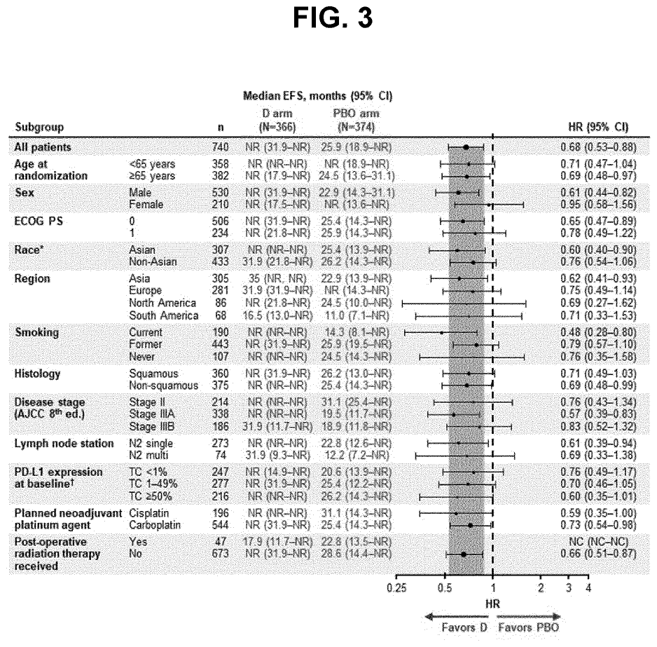

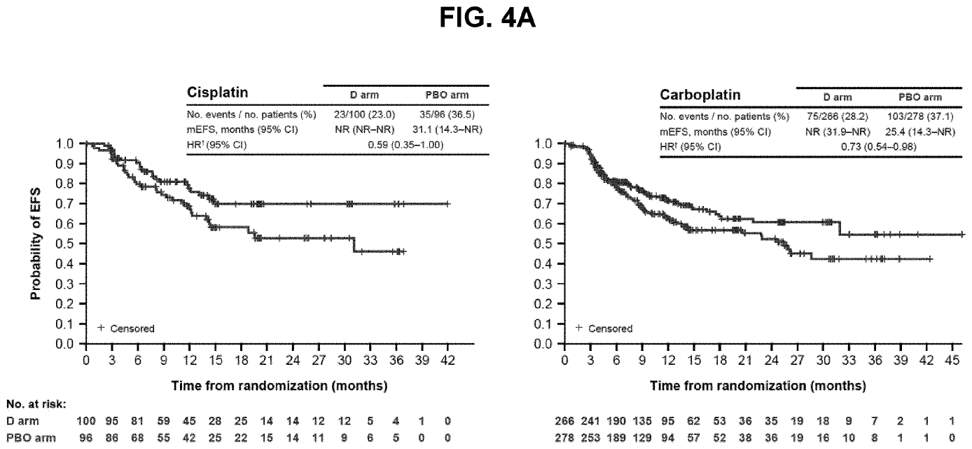

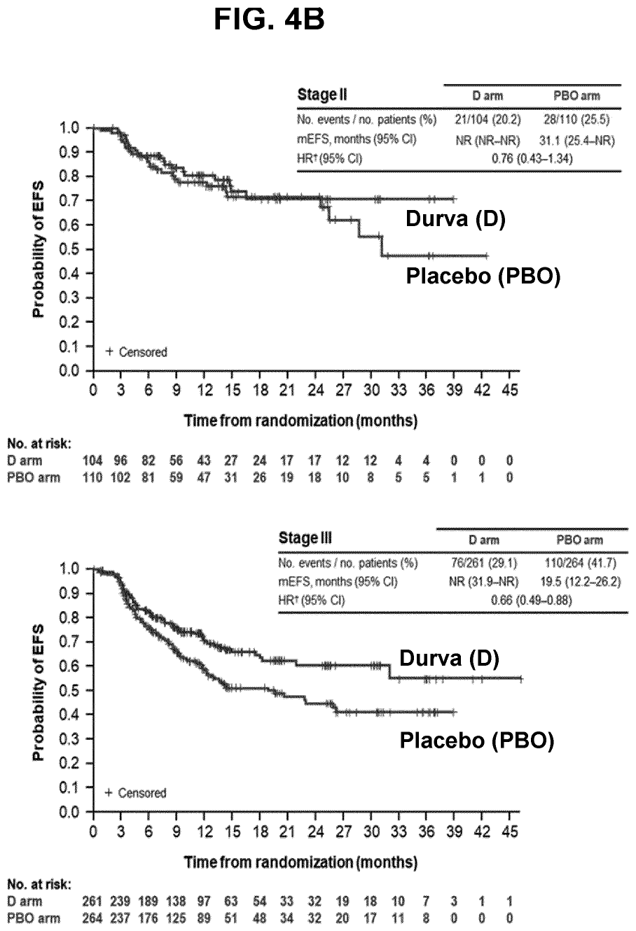

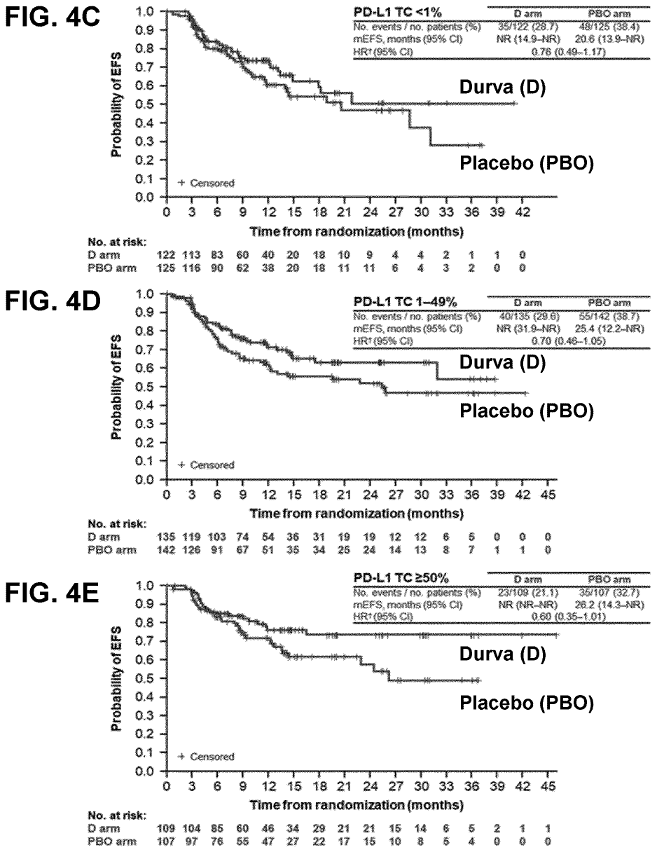

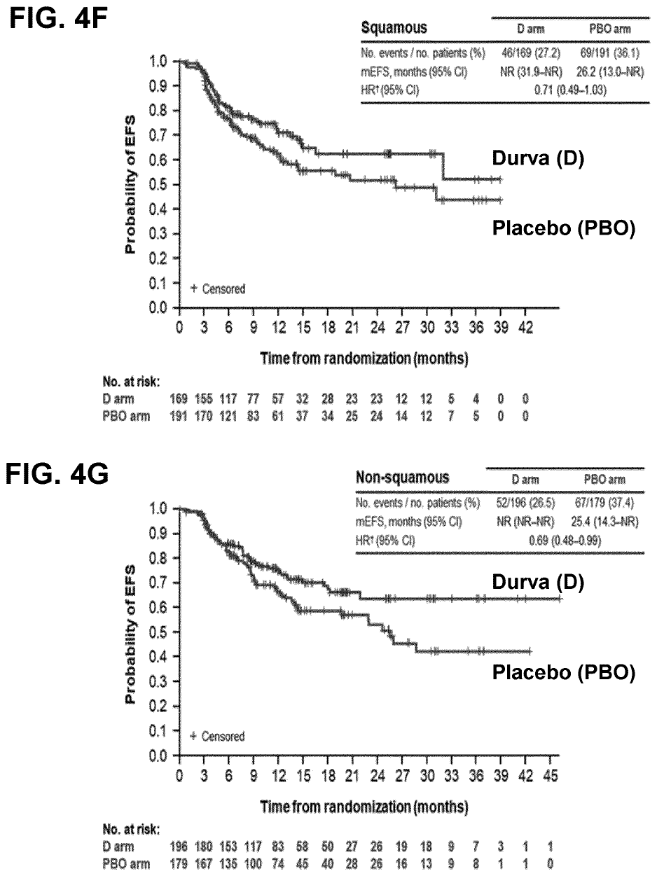

At this first interim analysis of EFS with 31.9% maturity, EFS was significantly prolonged in the durvalumab arm versus the placebo arm (stratified hazard ratio, 0.68; 95% CI, 0.53 to 0.88]; P=0.003902), with the median times not reached (NR) (95% CI, 31.9 months to NR) versus 25.9 months (95% CI, 18.9 to NR), respectively (FIG. 2). EFS benefit with durvalumab versus placebo was maintained across most prespecified subgroups (FIG. 3). Consistent benefit was observed regardless of the planned neoadjuvant platinum agent (FIG. 4). Benefit was also observed regardless of age, disease stage (FIG. 4B), PD-L1 tumor cell expression (FIG. 4C-4E), and tumor histology (FIG. 4F-4G). The magnitude of benefit was greater for current smokers. Female patients appeared to have a smaller treatment effect, but the HR CIs were wide for this subgroup, which comprised only 28.4% of the mITT population.

TABLE 11
Objective Response Prior to Surgery According to
Blinded Independent Central Review (RECIST v1.1)
in the Modified Intent-to-treat Population.

| | Durvalumab arm | | Placebo arm | |
|---|---|---|---|---|
| | (N = 366) | | (N = 374) | |
| Objective response rate | 206 | (56.3) | 142 | (38.0) |
| 95% CI | 51.0-61.4 | | 33.0-43.1 | |
| Patients with a response, n (%) | | | | |
| Complete response | 4 | (1.1) | 1 | (0.3) |
| Partial response | 202 | (55.2) | 141 | (37.7) |
| Patients with no response, n (%) | | | | |
| Stable disease | 124 | (33.9) | 189 | (50.5) |
| Progression | 11 | (3.0) | 15 | (4.0) |
| Not evaluable | 25 | (6.8) | 28 | (7.5) |

Data cut-off of Nov. 10, 2022 (N = 740). CI, confidence interval; RECIST v1.1, Response Evaluation Criteria in Solid Tumors version 1.1.

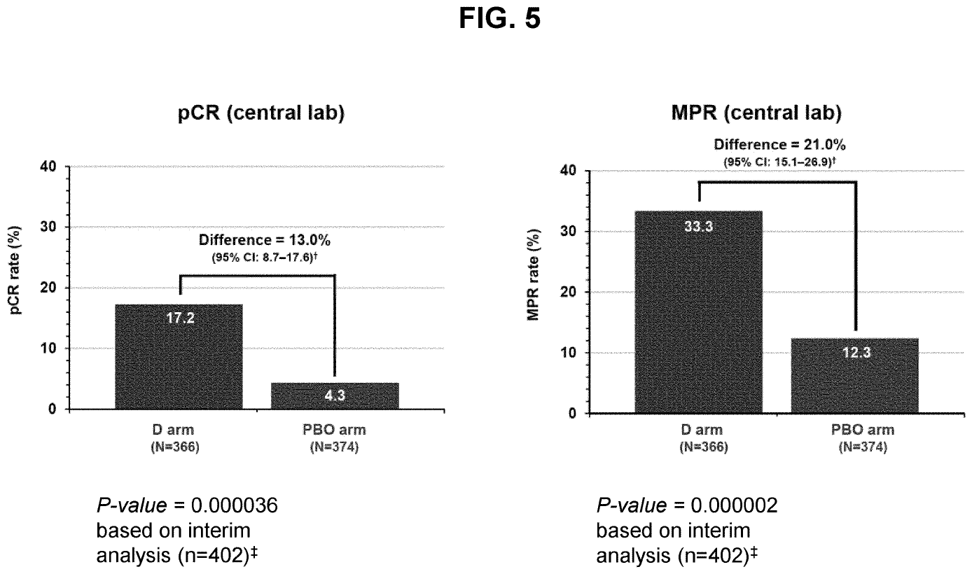

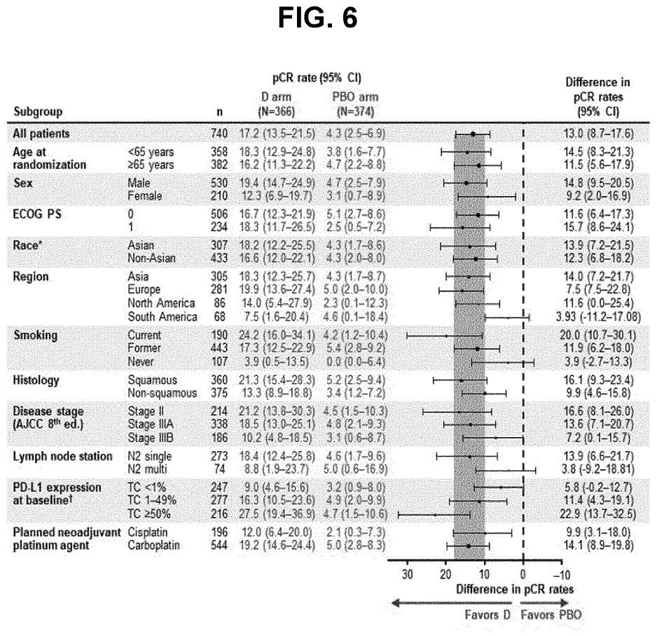

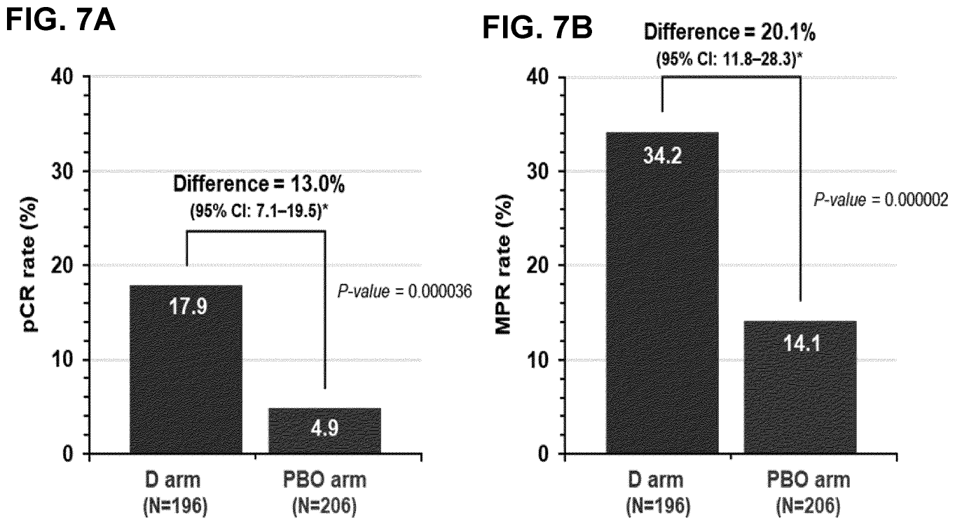

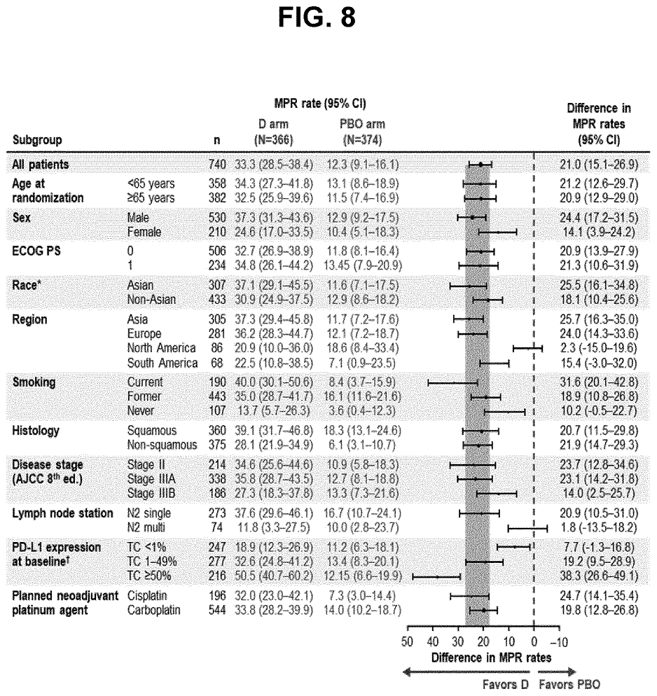

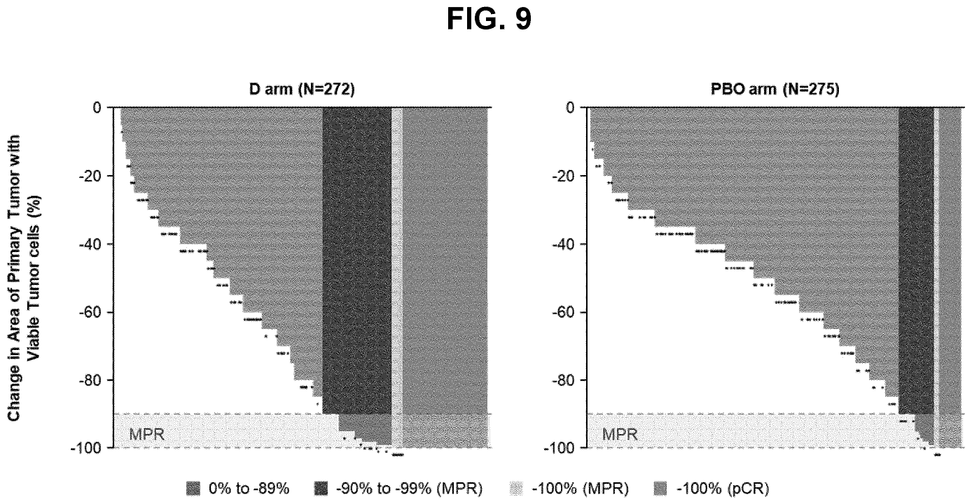

At the final analysis of pCR, a higher proportion of patients had achieved pCR in the durvalumab arm (17.2%; 95% CI, 13.5 to 21.5) versus the placebo arm (4.3%; 95% CI, 2.5 to 6.9) (difference in proportions, 13.0%; 95% CI, 8.7 to 17.6]) (FIG. 5). A higher proportion of patients also achieved MPR in the durvalumab arm (33.3%; 95% CI, 28.5 to 38.4) versus the placebo arm (12.3%; 95% CI, 9.1 to 16.1) (difference in proportions, 21.0; 95% CI, 15.1 to 26.9) (FIG. 5). Results for pCR and MPR were consistent at the interim analysis of pCR (N=402) (FIG. 7A-7B); statistical significance for pCR (P=0.0036) and MPR (P=0.000002) was achieved at this earlier analysis. A trend towards pCR benefit with durvalumab was observed across all prespecified subgroups, with the results largely consistent with the overall mITT population (FIG. 6). A similar trend was observed across subgroups for MPR (FIG. 8). Primary tumor pathological regression was, overall, greater in the durvalumab arm versus the placebo arm (FIG. 9). See Table 11 for a summary of objective response prior to surgery in the mITT population.

### Safety

Overall, adverse events (AEs) of any cause occurred in 96.5% of patients in the durvalumab arm and 94.7% of patients in the placebo arm (Table 5); AEs possibly related to any study treatment (i.e., durvalumab, placebo, or chemotherapy) occurred in 86.5% and 80.7%, respectively. The incidence of grade 3 or 4 AEs of any cause was similar between the study arms (42.3% vs. 43.4%); likewise, the incidence of grade 3 or 4 AEs possibly related to any study treatment was also similar (32.3% vs. 33.1%). AEs possibly related to any study treatment with an outcome of death were uncommon, with rates of 1.8% and 0.5% in the durvalumab and placebo arms, respectively. The most common AEs of any cause largely reflected the safety profile of the chemotherapy agents used in the study (Table 12); the rates of the most common AEs were largely similar across both treatment arms. Rates of any-grade rash (14% vs. 8.5%) and pruritus (11.8% vs. 5.5%) were higher among patients in the durvalumab arm versus the placebo arm; however, grade 3 or 4 events were uncommon, and occurred with similar frequency in both treatment arms. (See Table 13 for summary of the most common AEs possibly related to any study treatment.) Immune-mediated AEs of any grade were reported in 23.7% and 9.8% of patients in the durvalumab and placebo arms, respectively (Table 14); most were grade 1 or 2 with grade 3 or 4 events reported in 4.2% and 2.5% of patients, respectively. Immune-mediated pneumonitis of any grade was reported in 3.7% and 1.8% of patients in the durvalumab and placebo arms, respectively.

TABLE 12
Most Common Adverse Events of Any Cause in the Safety Analysis Set.

| | Durvalumab arm (N = 400) | | | | Placebo arm (N = 399) | | | |
|---|---|---|---|---|---|---|---|---|
| | | | Max. Grade | | | | Max. Grade | |
| | Any Grade | | 3 or 4 | | Any Grade | | 3 or 4 | |
| Event | Number of patients with event (%) | | | | | | | |
| Anemia | 136 | (34.0) | 26 | (6.5) | 126 | (31.6) | 26 | (6.5) |
| Nausea | 101 | (25.3) | 1 | (0.3) | 115 | (28.8) | 1 | (0.3) |
| Constipation | 99 | (24.8) | 1 | (0.3) | 85 | (21.3) | 0 | |
| Decreased appetite^{†} | 73 | (18.3) | 1 | (0.3) | 70 | (17.5) | 1 | (0.3) |
| Neutropenia | 68 | (17.0) | 36 | (9.0) | 71 | (17.8) | 38 | (9.5) |
| Alopecia | 68 | (17.0) | 0 | | 64 | (16.0) | 1 | (0.3) |
| Neutrophil count | 64 | (16.0) | 39 | (9.8) | 57 | (14.3) | 43 | (10.8) |
| decreased | | | | | | | | |
| Rash | 56 | (14.0) | 2 | (0.5) | 34 | (8.5) | 1 | (0.3) |
| Diarrhea | 52 | (13.0) | 3 | (0.8) | 49 | (12.3) | 3 | (0.8) |
| Fatigue | 52 | (13.0) | 0 | | 46 | (11.5) | 1 | (0.3) |
| Asthenia | 50 | (12.5) | 0 | | 54 | (13.5) | 5 | (1.3) |
| Pruritis | 47 | (11.8) | 1 | (0.3) | 22 | (5.5) | 0 | |
| Vomiting | 45 | (11.3) | 3 | (0.8) | 42 | (10.5) | 4 | (1.0) |
| Procedural pain | 44 | (11.0) | 1 | (0.3) | 48 | (12.0) | 2 | (0.5) |
| COVID-19^{‡} | 44 | (11.0) | 1 | (0.3) | 36 | (9.0) | 3 | (0.8) |

| | | | | | | |
|---|---|---|---|---|---|---|
| Insomnia | 41 | (10.3) | 0 | 46 | (11.5) | 0 |

Data cut-off of Nov. 10, 2022.
*The safety analysis set includes all randomized patients who received ≥1 dose of study treatment; adverse events were graded using Common Terminology Criteria for Adverse Events version 5.0. Included are adverse events reported with an any-grade incidence of at least 10% in the durvalumab arm during the overall study period, which spans from the first dose of study treatment (durvalumab or placebo or chemotherapy) until the earliest of: the last dose of study treatment or surgery + 90 days (taking the latest dose of durvalumab or placebo or chemotherapy or the date of surgery, +90 days); the data cut-off date; or the date of the first dose of subsequent anti-cancer treatment.
^{†}Two patients (one in each arm) had decreased appetite with an outcome of death (max. grade 5); the fatal event in the durvalumab arm was assessed as possibly related to study treatment by the investigator.
^{‡}Six patients had COVID-19 events of max. grade 5 (durvalumab arm, n = 5; placebo arm, n = 1); all COVID-19 deaths were assessed by the investigator as unrelated to study treatment (note: COVID-19 is summarized as a grouped term comprising the ‘COVID-19’ and ‘COVID-19 pneumonia’ preferred terms).

TABLE 13
Most Common Adverse Events Possibly Related to
Study Treatment in the Safety Analysis Set.

| | Durvalumab arm (N = 400) | | | | Placebo arm (N = 399) | | | |
|---|---|---|---|---|---|---|---|---|
| | | | Max. Grade | | | | Max. Grade | |
| | Any Grade | | 3 or 4 | | Any Grade | | 3 or 4 | |
| Event | Number of patients with event (%) | | | | | | | |
| Anemia | 104 | (26.0) | 17 | (4.3) | 96 | (24.1) | 20 | (5.0) |
| Nausea | 84 | (21.0) | 1 | (0.3) | 96 | (24.1) | 1 | (0.3) |
| Alopecia | 66 | (16.5) | 0 | | 58 | (14.5) | 1 | (0.3) |
| Neutrophil count decreased | 61 | (15.3) | 38 | (9.5) | 56 | (14.0) | 42 | (10.5) |
| Neutropenia | 60 | (15.0) | 36 | (9.0) | 64 | (16.0) | 35 | (8.8) |
| Decreased appetite^{†} | 49 | (12.3) | 0 | | 45 | (11.3) | 1 | (0.3) |
| Constipation | 44 | (11.0) | 0 | | 49 | (12.3) | 0 | |
| Fatigue | 42 | (10.5) | 0 | | 36 | (9.0) | 0 | |
| Rash | 37 | (9.3) | 2 | (0.5) | 27 | (6.8) | 1 | (0.3) |
| Hypothyroidism | 37 | (9.3) | 0 | | 6 | (1.5) | 0 | |
| Asthenia | 34 | (8.5) | 0 | | 38 | (9.5) | 0 | |
| Diarrhea | 34 | (8.5) | 3 | (0.8) | 21 | (5.3) | 3 | (0.8) |
| Leukopenia | 33 | (8.3) | 9 | (2.3) | 30 | (7.5) | 12 | (3.0) |
| Pruritis | 33 | (8.3) | 1 | (0.3) | 8 | (2.0) | 0 | |
| Vomiting | 31 | (7.8) | 3 | (0.8) | 28 | (7.0) | 4 | (1.0) |
| Alanine aminotransferase | 30 | (7.5) | 2 | (0.5) | 17 | (4.3) | 1 | (0.3) |
| increased | | | | | | | | |
| Platelet count decreased | 29 | (7.3) | 7 | (1.8) | 31 | (7.8) | 13 | (3.3) |
| White blood cell count | 25 | (6.3) | 8 | (2.0) | 32 | (8.0) | 12 | (3.0) |
| decreased | | | | | | | | |
| Thrombocytopenia | 24 | (6.0) | 6 | (1.5) | 29 | (7.3) | 9 | (2.3) |
| Arthralgia | 21 | (5.3) | 1 | (0.3) | 20 | (5.0) | 1 | (0.3) |

Data cut-off of Nov. 10, 2022.
*The safety analysis set includes all randomized patients who received ≥1 dose of study treatment; adverse events were graded using Common Terminology Criteria for Adverse Events version 5.0. Included are adverse events assessed by the investigator as possibly related to any study treatment (durvalumab or placebo or chemotherapy) reported with an any-grade incidence of at least 5% in the durvalumab arm during the overall study period, which spans from the first dose of study treatment until the earliest of: the last dose of study treatment or surgery + 90 days (taking the latest dose of durvalumab or placebo or chemotherapy or the date of surgery, +90 days); the data cut-off date; or the date of the first dose of subsequent anti-cancer treatment.
^{†}One patient in the durvalumab arm had decreased appetite with an outcome of death (grade 5) that was assessed as possibly related to study treatment by the investigator.

TABLE 14
Immune-mediated Adverse Events (Grouped Terms) Occurring in ≥1%
Patients in Either Treatment Arm in the Safety Analysis Set.*

| | Durvalumab Arm | | | | Placebo Arm | | | |
|---|---|---|---|---|---|---|---|---|
| | (N = 400) | | | | (N = 399) | | | |
| | | | Any Grade | | | | Any Grade | |
| | Any Grade | | 3 or 4 | | Any Grade | | 3 or 4 | |
| Any immune-mediated event, | 95 | (23.7) | 17 | (4.2) | 37 | (9.3) | 10 | (2.5) |
| n (%) | | | | | | | | |
| Hypothyroid events | 37 | (9.2) | 0 | | 9 | (2.3) | 0 | |
| Dermatitis/Rash | 22 | (5.5) | 2 | (0.5) | 7 | (1.8) | 1 | (0.3) |
| Pneumonitis | 15 | (3.7) | 5 | (1.2) | 7 | (1.8) | 4 | (1.0) |
| Hepatic events | 13 | (3.2) | 8 | (2.0) | 3 | (0.8) | 1 | (0.3) |

| Hyperthyroid events | 7 | (1.7) | 0 | 4 | (1.0) | 0 | |
|---|---|---|---|---|---|---|---|
| Diarrhea/Colitis | 3 | (0.7) | 0 | 5 | (1.3) | 3 | (0.8) |

Data cut-off of Nov. 10, 2022 (N = 799).
*The safety analysis set includes all randomized patients who received ≥1 dose of study Tx; AEs were graded using Common Terminology Criteria for Adverse Events v5.0. An immune-mediated AE was defined as an AE of special interest consistent with an immune-mediated mechanism of action, where there is no clear alternate etiology, and requiring the use of systemic corticosteroids or other immunosuppressants and/or, for specific endocrine events, endocrine therapy. AE, adverse event.

### Summary

Perioperative durvalumab plus neoadjuvant chemotherapy (CT) significantly improved both pCR and EFS among patients with resectable NSCLC versus neoadjuvant CT alone. The pCR rate was significantly higher in the durvalumab versus placebo arm (17.2% vs. 4.3% at final analysis; difference, 13.0%; 95% CI, 8.7 to 17.6; P=0.000036 assessed at interim analysis [n=402]). The improvement in pCR rate was 13.0% (95% CI: 8.7-17.6). The improvement in EFS was also significant (EFS stratified HR=0.68 (95% CI: 0.53-0.88; P=0.003902; median follow-up of 11.7 months and 31.9% maturity). Benefit was observed regardless of disease stage and PD-L1 expression.

Improvements in both pCR and EFS were largely consistent across predefined subgroups and EFS benefit was observed regardless of the planned neoadjuvant platinum agent (the HR was 0.59 (95% CI: 0.35-1.00) for cisplatin and 0.73 (95% CI: 0.54-0.98) for carboplatin).

Perioperative durvalumab+neoadjuvant CT was also associated with a manageable safety profile that was consistent with the known safety profiles of durvalumab and CT. Grade 3 or 4 any-cause adverse events occurred in 42.3% and 43.4%, respectively. The addition of durvalumab did not impact completion of neoadjuvant CT (4 cycles) or surgery

Perioperative durvalumab plus neoadjuvant chemotherapy significantly improved pCR and EFS versus neoadjuvant chemotherapy alone, with a manageable safety profile, in patients with R-NSCLC. This study is the first phase 3 study to describe the benefit of perioperative immunotherapy+neoadjuvant CT and demonstrate that perioperative durvalumab+neoadjuvant CT is a potential new treatment for patients with resectable NSCLC.

In patients with resectable NSCLC, perioperative durvalumab plus neoadjuvant chemotherapy, versus neoadjuvant chemotherapy alone, significantly improved both primary endpoints of EFS (HR, 0.68; P=0.003902) and pCR (difference in proportions, 13.0%; P=0.000036), with a manageable safety profile and no impact on completion of neoadjuvant chemotherapy or surgery. Notably, significant EFS benefit was achieved at the first planned interim analysis, based on 31.9% maturity and a median follow-up of less than 1 year (censored patients), when approximately a quarter of patients were still receiving adjuvant study treatment (as more than half of all patients were randomized in the last year of enrollment). These findings support an intensified treatment strategy with immunotherapy as a perioperative, rather than only a neoadjuvant or adjuvant, regimen.

EFS and pCR benefit with durvalumab was broadly observed across all predefined subgroups and was consistent with improvements observed in the overall mITT population. EFS benefit, for example, was observed regardless of age, disease stage (including patients with N2 disease), histology, and PD-L1 expression, including, notably, patients with PD-L1 expression<1% (although the magnitude of benefit was numerically better in patients with PD-L1 expression≥50%). A greater magnitude of benefit was observed in current smokers versus non-smokers; however, this is consistent with findings across other studies of immunotherapy (Zhao et al., “Impact of Smoking History on Response to Immunotherapy in Non-Small-Cell Lung Cancer: A Systematic Review and Meta-Analysis.” Front Oncol. 2021 Aug. 23; 11:703143. PMID: 34497760; PMCID: PMC8419340). Female patients appeared to derive less relative benefit; while the EFS HR confidence intervals were wide, overlapping with the male subgroup, this may have been due, at least in part, to the higher proportion of female patients who were never smokers (28.9% vs. 7.1%) and a lower proportion who had PD-L1 expression≥50% (24.6% vs. 32.1%), relative to male patients. While EFS and pCR benefit with durvalumab was observed regardless of disease stage, the magnitude somewhat varied, with stage II patients having a larger pCR benefit and stage IIIA patients (the largest subgroup) having a relatively larger EFS benefit. However, it is worth noting that, at this first interim analysis, there were fewer EFS events in the stage II subgroup, as these patients have a better prognosis, and many patients continued to received durvalumab or placebo at the time of the data cutoff. It is, therefore, likely that the full benefits from adjuvant durvalumab have not been fully realized in the current analysis, particularly for the stage II subgroup, and that additional maturity and follow-up is warranted to clearly assess EFS benefit, as well as overall survival and other longer-term secondary endpoints. Finally, a clear and consistent EFS benefit with durvalumab was observed regardless of whether the planned neoadjuvant chemotherapy regimen was cisplatin- or carboplatin-based; this underscores the efficacy of the broad variety of platinum-based regimens used in the neoadjuvant setting in real-world practice.

Use of perioperative durvalumab plus neoadjuvant chemotherapy in this study was associated with a manageable safety profile that was consistent with the known safety profiles of durvalumab and chemotherapy. The incidence of grade 3 or 4 AEs of any cause was similar between the study arms, occurring in 42.3% and 43.4% of patients in the durvalumab and placebo arms, respectively. AEs possibly related to study Tx with an outcome of death were rare in both arms. Immune-mediated AEs were more common in the durvalumab arm versus the placebo arm (23.5% vs. 9.8%); however, most were grade 1 or 2. Also, although differences in the populations and study designs of AEGEAN versus PACIFIC confound between-study comparisons (particularly, use of chemoradiotherapy in the latter), it is notable that the rates of any-grade and grade 3 or 4 immune AEs were similar between these studies (Antonia et al. N Engl J Med 2017; 377:1919-29; Antonia et al. N Engl J Med 2018; 379:2342-50). Finally, although AEGEAN enrolled patients during the height of the global Covid-19 pandemic, only 6 of 80 patients (7.5%) with any-grade Covid-19 had grade 5 Covid-19 events; in comparison, based on reports published before the widespread use of vaccines, patients with lung cancer were associated with Covid-19 mortality rates of 18-47% (Passaro et al. J Immunother Cancer 2021; 9:e002266).

Although comparison of the findings from this study (AEGEAN) with those of other studies that investigated immunotherapy as either neoadjuvant or adjuvant therapy may be considered informative, differences in patient populations and study designs confound cross-trial comparisons. AEGEAN is notable for several differences relative to other trials of immunotherapy in the resectable NSCLC setting (Forde et al. N Engl J Med 2022; 386:1973-85; Felip et al. Lancet 2021; 398:1344-57; O'Brien et al. Lancet Oncol 2022; 12:1274-86). Key strengths include: the large number of patients enrolled; the use of a double-blind, placebo-controlled design throughout both the neoadjuvant and adjuvant treatment periods; the exclusion of patients with documented EGFR/ALK aberrations from the pre-specified population for analyses of efficacy; the use of the 8^{th} edition of the AJCC staging manual; the use of the IASLC recommendations for assessment of pathological response; and the flexibility in neoadjuvant chemotherapy regimens. That said, AEGEAN was not designed to assess the individual contributions of the neoadjuvant and adjuvant components of treatment. Nonetheless, the results reported here justify a perioperative treatment strategy and substantiate other studies that have employed this approach with immunotherapy in the melanoma and NSCLC settings (Patel et al., N Engl J Med 2023; 388:813-823; Lu et al., Journal of Clinical Oncology 41, no. 36_suppl (Apr. 20, 2023) 425126-425126). Moreover, the benefit of the full course of perioperative durvalumab may only be fully realized until such time as all patients have had the opportunity to complete adjuvant study treatment.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present disclosure.

What is claimed is:

1: A method of treating a patient identified as having resectable non-small cell lung carcinoma (R-NSCLC), the method comprising administering to the patient a combination therapy comprising durvalumab and a platinum-based chemotherapy.

2: The method of claim 1, wherein the combination therapy is administered about every 14 to 28 days for about 3 to 6 cycles.

3: The method of either claim 1 or claim 2, wherein the combination therapy is administered about every 21 days (Q3W) for about 4 cycles.

4: The method of claim 2 further comprising resecting the R-NSCLC after the about 3 to 6 cycles of the combination therapy.

5: The method of claim 3 further comprising resecting the R-NSCLC after the about 4 cycles of the combination therapy.

6: The method of either claim 4 or claim 5 further comprising administering to the patient durvalumab about every 14 to 28 days for up to about twelve cycles after resecting the R-NSCLC.

7: The method of either claim 4 or claim 5 further comprising administering to the patient durvalumab about every 28 days (Q4W) for up to about twelve cycles after resecting the R-NSCLC.

8: The method of any one of claims 1-7, wherein the combination therapy comprises about 1000 to 2000 mg of durvalumab.

9: The method of any one of claims 1-8, wherein the combination therapy comprises about 1500 mg of durvalumab.

10: A method of treating a patient identified as having resectable non-small cell lung carcinoma (R-NSCLC), the method comprising
(i) administering to the patient about 1500 mg of durvalumab and a platinum-based chemotherapy about every 3 weeks (Q3W) for about 4 cycles;
(ii) removing the R-NSCLC by surgery; and then
(iii) administering to the patient about 1500 mg of durvalumab about every four weeks (Q4W) for at least about 12 weeks.

11: The method of any one of claims 1-10, wherein the platinum-based chemotherapy is one or more of carboplatin, cisplatin, nedaplatin, and oxaliplatin.

12: The method of claim 11, wherein the platinum-based chemotherapy comprises a carboplatin area under the serum drug concentration-time curve (AUC) dose of about 5 to about 6 mg/mL/min, or a cisplatin dose of about 75 mg/m².

13: The method of any one of claims 1-12, wherein the platinum-based chemotherapy further comprises one or more of afatinib, cetuximab, bevacizumab, erlotinib, gemcitabine, paclitaxel, pemetrexed, and vemurafenib.

14: The method of claim 13, wherein the platinum-based chemotherapy comprises a paclitaxel dose of about 200 mg/m², a pemetrexed dose of about 500 mg/m², or a gemcitabine dose of about 1250 mg/m².

15: The method of any one of claims 1-14, wherein the R-NSCLC is a squamous cell carcinoma.

16: The method of claim 15, wherein the platinum-based chemotherapy comprises:
(i) carboplatin and paclitaxel;
(ii) cisplatin and gemcitabine; and/or
(iii) carboplatin and gemcitabine.

17: The method of any one of claims 1-14, wherein the R-NSCLC is a non-squamous cell carcinoma.

18: The method of claim 17, wherein the platinum-based chemotherapy comprises:
(i) carboplatin and pemetrexed; and/or
(ii) cisplatin and pemetrexed.

19: The method of any one of claims 1-18, wherein the patient does not have an EGFR mutation and/or an ALK translocation.

20: The method of any one of claims 5-18, wherein resecting the R-NSCLC occurs within about 10 weeks after the about 3 to 6 cycles of the combination therapy.

21: The method of any one of claims 5-18 further comprising a post-operative radiation therapy.

22: The method of claim 21, wherein the post-operative radiation therapy starts within about 8 weeks after resecting the R-NSCLC.

23: The method of claim 22, wherein durvalumab starts within about 3 weeks from the end of the post-operative radiation therapy.

24: The method of any one of claims 1-23, wherein the method results in one or more of:
(i) improvement in event-free survival (EFS) of at least about 13 months;
(ii) improvement in pathological complete response (pCR) by at least about 13%; and
(iii) improvement in major pathological response (mPR) by at least about 21%.

25: The method of any one of claims 1-24, wherein the method results in improvement in one or more of EFS, pCR, mPR, DFS, and OS compared to a standard of care.

26: The method of any one of claims 1-24, wherein the method results in improvement in one or more of EFS, pCR, mPR, DFS, and OS compared to a platinum-based chemotherapy.

27: A combination therapy comprising durvalumab and a platinum-based chemotherapy for use in treating resectable non-small cell lung cancer (R-NSCLC) in a patient in need thereof.

28: The combination therapy for use of claim 27, wherein the combination therapy is administered about every 14 to 28 days for about 3 to 6 cycles.

29: The combination therapy for use of claim 27, wherein the combination therapy is administered about every 21 days (Q3W) for about 4 cycles.

30: The combination therapy for use of either claim 28 or claim 29 further comprising resecting the R-NSCLC after the about 3 to 6 cycles of the combination therapy.

31: The combination therapy for use of either claim 28 or claim 29 further comprising resecting the R-NSCLC after the about 4 cycles of the combination therapy.

32: The combination therapy for use of either claim 30 or claim 31 further comprising administering to the patient durvalumab about every 14 to 28 days for up to about twelve cycles after resecting the R-NSCLC.

33: The combination therapy for use either claim 30 or claim 31 further comprising administering to the patient durvalumab about every 28 days (Q4W) for up to about twelve cycles after resecting the R-NSCLC.

34: The combination therapy for use of any one of claims 27-33, wherein the combination therapy comprises about 1000 to 2000 mg of durvalumab.

35: The combination therapy for use of any one of claims 27-34, wherein the combination therapy comprises about 1500 mg of durvalumab.

36: A combination therapy for use in treating a patient identified as having resectable non-small cell lung carcinoma (R-NSCLC), wherein the combination therapy comprises:
(i) administering to the patient about 1500 mg of durvalumab and a platinum-based chemotherapy about every 3 weeks (Q3W) for about 4 cycles; and then
(ii) administering to the patient about 1500 mg of durvalumab about every four weeks (Q4W) for at least about 12 weeks after removal of the R-NSCLC by surgery.

37: The combination therapy for use of any one of claims 27-36, wherein the platinum-based chemotherapy is one or more of carboplatin, cisplatin, nedaplatin, and oxaliplatin.

38: The combination therapy for use of claim 37, wherein the platinum-based chemotherapy comprises a carboplatin area under the serum drug concentration-time curve (AUC) dose of about 5 to about 6 mg/mL/min, or a cisplatin dose of about 75 mg/m².

39: The combination therapy for use of any one of claims 27-38, wherein the platinum-based chemotherapy further comprises one or more of afatinib, cetuximab, bevacizumab, erlotinib, gemcitabine, paclitaxel, pemetrexed, and vemurafenib.

40: The combination therapy for use of claim 39, wherein the platinum-based chemotherapy comprises a paclitaxel dose of about 200 mg/m², a pemetrexed dose of about 500 mg/m², or a gemcitabine dose of about 1250 mg/m².

41: The combination therapy for use of any one of claims 27-40, wherein the R-NSCLC is a squamous cell carcinoma.

42: The combination therapy for use of claim 41, wherein the platinum-based chemotherapy comprises:
(i) carboplatin and paclitaxel;
(ii) cisplatin and gemcitabine; and/or
(iii) carboplatin and gemcitabine.

43: The combination therapy for use of any one of claims 27-40, wherein the R-NSCLC is a non-squamous cell carcinoma.

44: The combination therapy for use of claim 43, wherein the platinum-based chemotherapy comprises:
(i) carboplatin and pemetrexed; and/or
(ii) cisplatin and pemetrexed.

45: The combination therapy for use of any one of claims 27-44, wherein the patient does not have an EGFR mutation and/or an ALK translocation.

46: The combination therapy for use of any one of claims 31-45, wherein resecting the R-NSCLC occurs within about 10 weeks after the about 3 to 6 cycles of the combination therapy.

47: The combination therapy for use of any one of claims 31-46 further comprising a post-operative radiation therapy.

48: The combination therapy for use of claim 47, wherein the post-operative radiation therapy starts within about 8 weeks after resecting the R-NSCLC.

49: The combination therapy for use of claim 48, wherein durvalumab starts within about 3 weeks from the end of the post-operative radiation therapy.

50: The combination therapy for use of any one of claims 27-49, wherein the use of the combination therapy results in one or more of:
(i) improvement in event-free survival (EFS) of at least about 13 months;
(ii) improvement in pathological complete response (pCR) by at least about 13%; and
(iii) improvement in major pathological response (mPR) by at least about 21%.

51: The combination therapy for use of any one of claims 27-50, wherein the use of the combination therapy results in improvement in one or more of EFS, pCR, mPR, DFS, and OS compared to a standard of care.

52: The combination therapy for use of any one of claims 27-50, wherein the use of the combination therapy results in improvement in one or more of EFS, pCR, mPR, DFS, and OS compared to a platinum-based chemotherapy.

53: The use of a combination therapy comprising durvalumab and a platinum-based chemotherapy for the manufacture of a medicament for treating resectable non-small cell lung cancer (R-NSCLC) in a patient in need thereof.

54: The use of claim 53, wherein the medicament is administered about every 14 to 28 days for about 3 to 6 cycles.

55: The use of claim 53, wherein the medicament is administered about every 21 days (Q3W) for about 4 cycles.

56: The use of either claim 54 or claim 55 further comprising resecting the R-NSCLC after the about 3 to 6 cycles of the combination therapy.

57: The use of either claim 54 or claim 55 further comprising resecting the R-NSCLC after the about 4 cycles of the combination therapy.

58: The use of either claim 56 or claim 57 further comprising administering to the patient durvalumab about every 14 to 28 days for up to about twelve cycles after resecting the R-NSCLC.

59: The use of either claim 56 or claim 57 further comprising administering to the patient durvalumab about every 28 days (Q4W) for up to twelve cycles after resecting the R-NSCLC.

60: The use of any one of claims 53-59, wherein the medicament comprises about 1000 to 2000 mg of durvalumab.

61: The use of any one of claims 53-60, wherein the medicament comprises about 1500 mg of durvalumab.

62: The use of a durvalumab and a platinum-based chemotherapy in the manufacture of a medicament for treating resectable non-small cell lung cancer (R-NSCLC) in a combination therapy, wherein the combination therapy comprises:
(i) administering to the patient about 1500 mg of durvalumab and a platinum-based chemotherapy about every 3 weeks (Q3W) for about 4 cycles;
(ii) removing the R-NSCLC by surgery; and then
(iii) administering to the patient about 1500 mg of durvalumab about every four weeks (Q4W) for at least about 12 weeks.

63: The use of any one of claims 53-62, wherein the platinum-based chemotherapy is one or more of carboplatin, cisplatin, nedaplatin, and oxaliplatin.

64: The use of claim 63, wherein the platinum-based chemotherapy comprises a carboplatin area under the serum drug concentration-time curve (AUC) dose of about 5 to about 6 mg/mL/min, or a cisplatin dose of about 75 mg/m².

65: The use of any one of claims 53-64, wherein the platinum-based chemotherapy further comprises one or more of afatinib, cetuximab, bevacizumab, erlotinib, gemcitabine, paclitaxel, pemetrexed, and vemurafenib.

66: The use of claim 65, wherein the platinum-based chemotherapy comprises a paclitaxel dose of about 200 mg/m², a pemetrexed dose of about 500 mg/m², or a gemcitabine dose of about 1250 mg/m².

67: The use of any one of claims 53-66, wherein the R-NSCLC is a squamous cell carcinoma.

68: The use of claim 67, wherein the platinum-based chemotherapy comprises:
(i) carboplatin and paclitaxel;
(ii) cisplatin and gemcitabine; and/or
(iii) carboplatin and gemcitabine.

69: The use of any one of claims 53-66, wherein the R-NSCLC is a non-squamous cell carcinoma.

70: The use of claim 69, wherein the platinum-based chemotherapy comprises:
(i) carboplatin and pemetrexed; and/or
(ii) cisplatin and pemetrexed.

71: The use of any one of claims 53-70, wherein the patient does not have an EGFR mutation and/or an ALK translocation.

72: The use of any one of claims 53-71, wherein resecting the R-NSCLC occurs within about 10 weeks after the about 3 to 6 cycles of the combination therapy.

73: The use of any one of claims 53-72 further comprising a post-operative radiation therapy.

74: The use of claim 73, wherein the post-operative radiation therapy starts within about 8 weeks after resecting the R-NSCLC.

75: The use of claim 74, wherein durvalumab starts within about 3 weeks from the end of the post-operative radiation therapy.

76: The use of any one of claims 53-75, wherein the use of the combination therapy results in one or more of:
(i) improvement in event-free survival (EFS) of at least about 13 months;
(ii) improvement in pathological complete response (pCR) by at least about 13%; and
(iii) improvement in major pathological response (mPR) by at least about 21%.

77: The use of any one of claims 53-76, wherein the use of the combination therapy results in improvement in one or more of EFS, pCR, mPR, DFS, and OS compared to a standard of care.

78: The use of any one of claims 53-76, wherein the use of the combination therapy results in improvement in one or more of EFS, pCR, mPR, DFS, and OS compared to a platinum-based chemotherapy.
